(19) United States
(12) Patent Application Publication
(10) Pub. No.: US 2025/0127807 A1
(43) Pub. Date: Apr. 24, 2025

(54) TREATMENT OF VAGINAL DYSBIOTIC CONDITIONS WITH ASSOCIATED ELEVATED VAGINAL CALPROTECTIN

(71) Applicant: Gary A. GELBFISH, Brooklyn, NY (US)

(72) Inventor: Gary A. GELBFISH, Brooklyn, NY (US)

(21) Appl. No.: 18/834,994

(22) PCT Filed: Feb. 2, 2023
(86) PCT No.: PCT/US2023/012223

§ 371 Date: Jul. 31, 2024

Related U.S. Application Data
(60) Provisional application No. 63/305,996, filed on Feb. 2, 2022.

(51) Int. Cl. A61K 33/32; A61K 9/00; A61K 33/30; A61K 35/00; A61K 35/747; A61K 38/17; A61K 38/40; A61P 15/02; G01N 33/68; G01N 33/84

(52) U.S. Cl. CPC A61K 33/32; A61K 9/0034; A61K 33/30; A61K 35/747; A61K 38/1709; A61K 38/40; A61P 15/02; G01N 33/6893; G01N 33/84; A61K 2035/115; G01N 2800/36

(57) ABSTRACT

Vaginal dysbiotic conditions accompanied by elevated levels of calprotectin, such as bacterial vaginosis and preliminary conditions that can lead to bacterial vaginosis, are treated with intravaginal administration of essential metal of manganese, zinc or manganese and zinc. to partially or fully counteract binding capacity for those metals by the calprotectin, permitting manganese to become more available to support establishment of a healthy vaginal microbiome dominated by beneficial lactobacilli to correct the dysbiotic condition.

### CROSS-REFERENCE TO RELATED APPLICATIONS

This application claims benefit of U.S. provisional patent application No. 63/305,996 entitled “TREATMENT OF VAGINAL DYSBIOTIC CONDITIONS WITH ASSOCIATED ELEVATED VAGINAL CALPROTECTIN” filed Feb. 2, 2022, the entire contents of which are incorporated by reference here for all purposes.

This application is related subject matter to international patent application number PCT/US2021/044076 filed Jul. 30, 2021 (the U.S. national stage of which has been assigned U.S. patent application Ser. No. 18/017,239), which claims priority to U.S. provisional patent application No. 63/059,817 filed Jul. 31, 2020, the entire contents of each of which are incorporated by reference herein for all purposes.

### FIELD OF THE INVENTION

The invention relates to treatment of vaginal dysbiotic conditions, such as bacterial vaginosis or pre-bacterial vaginosis, with associated elevated vaginal calprotectin.

### BACKGROUND OF THE INVENTION

Bacterial vaginosis (BV) is the leading vaginal disorder in women of childbearing age. Women with BV are at increased risk for acquiring of several sexually transmitted infections (STIs) including HIV, Neisseria gonorrhoeae, Chlamydia trachomatis, and herpes simplex virus 2, as well as complications after gynecologic surgery, and recurrence of BV itself. BV also increases the risk of HIV transmission to male sex partners. BV during pregnancy is an established risk factor for spontaneous abortion, preterm delivery and associated neonatal complications. Even for infants born at term, an increased risk of neonatal assisted ventilation/respiratory distress, admission to neonatal intensive care units, and neonatal sepsis was found in BV-exposed infants. Not all patients with BV are symptomatic. However, the Centers for Disease Control recommends that all symptomatic women should be treated for BV to relieve symptoms and signs of infection and to reduce the risk of acquiring and spreading HIV and other STIs.

BV is characterized by a profound shift in the normal vaginal microbiota, from lactobacilli in a low-pH environment that are replaced with multiple pathogenic species (e.g. Gardnerella vaginalis, Atopobium vaginae and Prevotella species) in an elevated pH environment. The lining of the human female vagina is normally inhabited by several Lactobacilli species that produce lactate and maintain a low pH environment. Normally, these bacteria provide a protective barrier against infection by undesirable bacteria which cannot live in the relatively acidic environment. These beneficial lactobacilli are described scientifically as the “normal vaginal microbiome.” A disturbance of this normal microbiome is the hallmark of BV. Symptomatic and asymptomatic BV are estimated to be highly prevalent in various populations with some studies showing one or more episodes in 29% of all women aged 14 to 49 years and in 50% of African American women.

Currently approved, standard-of-care treatments for BV all rely on antibiotics targeting pathogenic microbes, and all show unacceptably low cure rates and high relapse rates. While antibiotics are typically successful to provides relief of symptoms, the relief is most often temporary, and recurrence is common, with an approximately 50% recurrence within 6 months. There have been many efforts to impact this vicious cycle of treatment and recurrence, but without significant success to date. The limitations of this unidimensional antibiotic-based approach to such a complex condition are well known.

Other approaches to BV treatment include vaginal probiotics, vaginal pH manipulation, metal chelation compounds, DNases for biofilm disruption, simultaneous treatment of sexual partners with antibiotics or condom use to avoid reinfection, and contraceptive hormonal intervention. None of these interventions has proved a reliable, sustained treatment for BV.

There is a significant need for improved treatments for BV and to address the current high incidence of BV recurrence.

### SUMMARY OF INVENTION

The inventor has identified treatments for vaginal dysbiotic conditions with impairment of beneficial lactobacilli, such as bacterial vaginosis (BV) or transitory dysbiotic conditions preliminary to bacterial vaginosis, that address an observed significant increase in calprotectin levels associated with BV and clear trend of increasing levels of calprotectin (CP) correlated to the progressive deterioration of the vaginal microbiome from normal towards BV.

A critical starting presumption of this work was that the baseline vaginal nutritional environment may be a selective force in determining the microbiome i.e., that there is a conducive environment for lactobacilli growth as compared to another environment that is conducive for the growth of BV associated bacteria. From that presumption, it follows that at some point in time, a changed vaginal environment develops that now is no longer conducive to the growth of beneficial lactobacilli. While it is well known that bacteria themselves actively attempt to exclude other bacteria via their modification of the environment via lactate/pH, hydrogen peroxide, biogenic amines, bacteriocins etc., a focus of the present invention is on the body's apparent active contribution to this process of environmental modification.

The observed increase in calprotectin levels in BV and correlation of calprotectin levels to progression of vaginal microbiome toward BV has not, to the inventor's knowledge, been previously described and is unexpected considering the noted paucity of white blood cells (WBC) typically seen in the vaginal fluid in women with BV. It is consistent with the known inflammatory profiles that have been recently shown to be elevated in BV, including IL-1beta and IL-6, despite the marked low level of local WBC. It is shown that relatively consistent levels of manganese (Mn) and zinc (Zn) in women with and without BV indicate a steady supply of these critical nutrients. Of great note, however, is the observation that the calprotectin levels seen in BV are sufficiently high to completely bind all the Mn and Zn present in vaginal fluid. This appears to be a classic manifestation of nutritional immunity and is expected to create a manganese and zinc starvation condition; where one did not exist before BV occurred. As such, the inventor has identified that the vaginal environment available for bacteria to grow is fundamentally changed by the body. Furthermore, this condition should prevail as long as increased calprotectin levels are maintained. While the discovery of increased calprotectin levels in BV may be interesting, why should Mn and Zn restriction negatively impact the normal microbiome to a greater extent than the BV microbiome?

To answer this question, one must first understand the selective mineral needs of bacteria in general which are known to be variable and the specific needs of some, but not all lactobacilli. While it is common knowledge that as a general rule bacteria need iron, it is less known that most lactobacilli species are an exception to this rule and instead utilize Mn. Lactobacilli are one of only two known exceptions; the other exception is Borrelia burgdorferi, the bacteria responsible for Lyme disease. These unusual bacteria have rearranged their metabolic machinery and use manganese instead of iron for electron transfer and other enzymatic and redox needs. Indeed, DeMan Rogosa and Sharpe (MRS) agar is a lactobacillus selective growth media which utilizes high levels of manganese and relatively low levels of iron to both encourage growth of lactobacilli and suppress growth of other bacteria.

What is further noteworthy about Mn is that it is a key component of some lactobacilli species' capacity to neutralize toxic oxygen radicals. This permits many lactobacilli to create and thrive in a low pH, oxidative environment that is toxic to most other bacteria including even some lactobacillus species. Mn functions as an integral component of some forms of Superoxide Dismutase (SOD), an enzyme that catalyzes the dismutation of superoxide radicals (O2-) to molecular oxygen (O2) and hydrogen peroxide (H2O2). When present in sufficient quantities, manganese may act alone to neutralize superoxide if SOD is not present. This phenomenon is well documented in L. plantarum where this species lacks SOD and requires much higher levels of Mn.

The ability of beneficial Lactobacilli to survive in oxidative environments is also consistent with high Oxidation Reduction Potential (ORP) levels found in vaginal fluid in those with a normal microbiome, which may be on the order of approximately +300 mV, as compared to an average decrease of approximately −250 mV for those with BV. This low ORP returns to normal after successful treatment of BV.

The vaginal environment is usually conducive to lactobacilli since, as the inventor has observed, it provides a stable and plentiful source of Mn and Zn. By utilizing manganese instead of iron, lactobacilli obviate the need to possess complex iron scavenging systems that would otherwise be needed.

As is well documented, Lactobacilli do not elicit an inflammatory response. As homofermentive organisms they produce hydrogen peroxide and lactate which lowers the pH and provides an environment that becomes even less conducive to invading BV associated bacteria as compared to what was initially created by the body. All these characteristics coalesce to provide a balanced symbiotic microbiome that is well suited for its vaginal niche and is the norm for the majority of women during their entire reproductive years. Non-limiting examples of beneficial D-lactic acid-producing Lactobacilli include: L. crispatus, L. jensenii, L. johnsonii, L. gasseri, L. helveticus, L. rhamnosus, L. reuteri, L. casei, and L. plantarum.

Not to be bound be theory, but to aid in the understanding of the invention, the inventor hypothesizes that the maintenance of a healthy, lactobacillus-dominated vaginal microbiome, is predicated on there being no activation of the body's nutritional immunity system with the associated production of high levels of calprotectin. Infection with certain bacteria, or other mucosal insults may initiate an inflammatory condition, which leads to an increase in vaginal concentrations of calprotectin resulting in manganese starvation of the resident beneficial lactobacilli as a consequence of sequestration of manganese by calprotectin. When this occurs, the Mn-starved lactobacilli's capacity to produce lactate and hydrogen peroxide progressively deteriorates, and the ability of the beneficial lactobacilli to live in a stressed, low pH, high ORP environment is reduced when they have insufficient levels of Mn available to them. As the pH rises, the environment becomes non-exclusive and other, pathogenic bacteria gain a foothold. As BV associated bacteria thrive, inflammation increases, further contributing to elevation of calprotectin. This vicious circle ensures a continued manganese starvation environment and the beneficial lactobacilli being chronically displaced as the dominant bacteria. This state of affairs will continue as long chronic inflammation persists and calprotectin levels remain significantly elevated.

Some bacteria are known to source trace metals even from high-affinity metalloproteins. Gardnerella Vaginalis has been shown to have surface proteins that can directly bind and extract iron from human lactoferrin. Neisseria Gonorrhea has been found to scavenge Mn from calprotectin and iron from lactoferrin. This presumably contributes to its virulence. The ability of lactobacilli to scavenge Mn from calprotectin, however, has never been shown.

The hypothesized theoretical framework explains the resistance of the BV microbiome-once established, to revert to its previous lactobacillus-based microbiome. This framework may be useful to understand the relative failure of other well studied BV treatments including antibiotics, probiotics, EDTA metal chelation and lactate supplementation.

In this framework, unless entirely effective, antibiotics would not be expected to change the underlying inflammatory environment since regrowth of L. crispatus and other beneficial lactobacilli appears to be the only way to eliminate this low level inflammation that leads to elevated calprotectin levels. Indeed, after antibiotic treatment, the most common microbiome is an L. iners dominant microbiome, but L. iners has similar inflammatory activity to G. vaginalis, in contradistinction to L. crispatus. Thus L. iners would be expected to perpetuate the inflammatory/calprotectin cycle.

Some have tried exogenous supplied probiotic bacteria with limited success. The theoretical framework summarized above would predict such failure since one would not expect the bacteria to thrive and overwhelm BV associated bacteria. Why should the newly introduced bacteria fare any better than the original beneficial bacteria in the modified vaginal environment unless manganese starvation from elevated calprotectin is addressed?

Others have tried chelation therapies such as EDTA. The theoretical framework presented would predict that chelation therapies that bind multiple metals including Mn would only exacerbate the trace-metal deficiency that exists secondary to lactoferrin and calprotectin.

Lastly, lowering the pH via lactate may change the environment vis-a-vis other species that grow poorly in low pH environments but would not be expected to encourage growth of beneficial vaginal lactobacilli since low pH is ultimately a negative feedback loop for healthy lactobacillus growth, and would depress the beneficial lactobacilli in a low Mn environment-since their ability to live in oxidative stress environments without Mn is compromised.

The theoretical framework is supported by testing that has been performed, summarized in the examples section below, and has led to the identification of the proposed therapeutic treatments of the present invention that is compatible with this nutritional immunity framework. A prime component of a therapeutic treatment of the invention is to increase availability of essential metals, such as Mn and/or Zn, to support proliferation of beneficial lactobacilli in the presence of elevated vaginal calprotectin levels. Sufficient Mn and/or Zn may be applied locally, topically and in a form that totally overwhelms the calprotectin binding sites and thereby making both these nutrients available to beneficial lactobacilli. The amount of Mn needed for optimal growth of a given bacteria is highly dependent on particular species and environment and will need to be studied further, but by counteracting the essential metal binding effect of elevated calprotectin, normally available levels of Mn and Zn should again become available for use by the beneficial lactobacilli. Additional supplemental Mn and/or Zn may also be provided to impart an environment even richer in Mn and Zn to further promote re-establishment of a vaginal microbiome dominated by beneficial lactobacilli. Ultimately, enough Mn and Zn needs to be provided to the bacteria for their particular metabolic needs, thus contravening the body's effort to suppress them.

Another possibility is to reduce the body's inflammatory state in general or locally. For example, anti-inflammatory agents can be given systemically or applied locally. This could suppress calprotectin production.

Bovine lactoferrin is known to be anti-inflammatory, in part via binding bacterial lipopolysaccharide (endotoxin.) Bovine-lactoferrin can be used topically in conjunction with Zn and Mn supplementation. A second effect of bovine lactoferrin is iron chelation. Bovine lactoferrin is specifically bacteriostatic to bacteria who need iron. As described above, many bacteria have adapted to extract iron from human lactoferrin via specialized proteins. However, extraction of iron from bovine-lactoferrin by bacterial species that are seen in the human genital tract is unlikely since it would require different proteins as compared to extraction from human-lactoferrin. This is because human and bovine lactoferrin are only 70% homologous in their protein sequences. Such cross-species protein-binding functionality is highly unusual. In the case of beneficial lactobacilli, iron starvation should be a selective force since it should have little effect on them, since their metabolism is Mn based. It should, however, suppress the usual BV associated bacteria, which need iron for bacterial function.

Besides control of inflammation to decrease calprotectin levels, a specific calprotectin binder could be developed to limit its effectiveness.

This multimodality therapy can be further expanded to include pH modification modalities or even oxidants such as urea peroxide; to mimic normal beneficial bacteria's strategies to suppress harmful bacteria. As understanding predicts, however, any such treatment will only be non-toxic to beneficial lactobacilli, once their environmental/nutritional needs of Mn and Zn are met so that their own mechanisms of dealing with oxidate stress are not disabled. Otherwise, these attempts to treat BV will also suppress Mn-starved lactobacilli.

Lastly, specific sugars that are selectively utilized by beneficial lactobacilli (such as sucrose, lactose or others) as a carbohydrate source and not by BV associated bacteria can be added to essentially create an artificial vaginal culture media that is additionally conducive to beneficial lactobacilli. Therapy would continue until beneficial bacteria are able to grow and restore their environment to one that is non-inflammatory and conducive to their own growth but not conducive to other bacteria that cannot live in a low pH and high oxidative environment that beneficial lactobacilli typically create.

The methods of treatment of the invention are also compatible with antibiotic or probiotic modalities since an enhanced environment is necessary to the recovery of a beneficial microbiome, whether it is facilitated by the initial destruction of harmful bacteria via antibiotics, of the seeding of beneficial bacteria or a combination of both. In either case, the few remaining beneficial endogenous bacteria or exogenously introduced probiotics would have an easier time to gain a foothold in a more conducive environment. Some or all of the above-described components such as Mn and Zn supplementation, lactoferrin, oxidants, sugars etc. can be co-administered with probiotics. As no animal models exists for BV, even in primates, only human trials can generate the appropriate data to determine the most beneficial levels of replacement therapy and associated modalities for an optimal BV treatment regimen.

Besides the treatment of BV and to enhance the recovery of a normal microbiome, the methods of treatment of the invention are highly likely to be useful in, and are also targeted for treatment of, transitional microbiota, a dysbiotic condition where the microbiome is clearly abnormal, but it has not fully descended into frank BV. Such a transitional microbiota condition is at times generally referred to herein as a pre-bacterial vaginosis condition, which includes intermediate BV. This would be useful since it has been conclusively shown that a transitional microbiome makes the occurrence of BV more likely.

L. iners is a well-established bacterial species of the transitional microbiome. L. iners is an unusual lactobacillus in that, similar to other lactobacilli, it is resistant to the typical antibiotic treatment of BV, yet it has iron needs that are more similar to BV associated bacteria. Furthermore, L. iners produces L-lactate in contrast to D-lactate producing beneficial lactobacilli. It is frequently seen in vaginal environments of abnormally high pH. When BV occurs, its relative numbers are lowered secondary to the multispecies bacterial overgrowth seen in BV, yet because of its antibiotic resistance, it often emerges as the dominant bacteria after the antibiotic treatment of BV. Current CDC recommendations are not to treat the transitional microbiome. This current work presented herein on understanding BV leads one to question this recommendation.

As described above, oxygen toxicity and redox environment is an issue for all bacteria especially anaerobes, yet some bacteria especially many lactobacilli are not strict anaerobes. They are aerotolerant, in that they can exist in aerobic conditions as well. This is because they have developed mechanisms to deal with oxidative stress. Mn, unlike iron, in sufficient intra-bacterial levels acts as an antioxidant in some lactobacilli. In other lactobacilli, Mn is a component of the superoxide dismutase (SOD) enzyme. Their metabolic adaptation permits them to survive in a relatively toxic environment that they create via a low pH and hydrogen peroxide. This successfully excludes other bacteria that do not have oxidative protection from the vaginal microbiome. Indeed, this mechanism is the reason for the relatively low bacterial diversity seen in the human vagina as compared to other human microbiomes of the mouth, skin, and colon.

The inventor has found that besides L. iners being iron dependent and not Mn dependent, a key additional difference of L. iners is that it has relatively poor tolerance to oxidative stress as evidenced by its inability to grow aerobically despite high levels of all pertinent minerals. This explains the observation of why L. iners is seldom present in robust L. crispatus microbiomes that create a low pH, oxidative environment. On the other hand, when the environment deteriorates because beneficial bacteria are scarce or not metabolically active, L. iners does grow. Under these circumstances, L. iners is actually in a privileged position since it can grow even in a Mn deficient environment. L. iners will grow provided that the oxygen tension is sufficiently low as normally exists in the vagina.

So, while L. iners has always been noted more frequently in abnormal microbiomes, the microbiology findings presented herein explain why this is so. L. iners is an apparent indicator of a deteriorating microbiome that no longer produces sufficient lactate and hydrogen peroxide to exclude L. iners and other potentially harmful bacteria. Sort of a reverse canary in the coal mine. For if the beneficial bacterial were functioning well, L. iners would not be able to grow since it lacks a high level of oxidative protection. As mentioned, the association of L. iners with a transitional microbiome has not previously led to a recommendation for its treatment. This is expected since a direct causative link between L. iners and BV had not previously been established.

This original work of the inventor leading to the present invention shows a clear correlation of L. iners-dominant microbiomes and elevated calprotectin levels, albeit not to the levels seen in established BV. Regardless, the findings show that beyond it being just an indicator of a malfunctioning microbiome, L. iners is an active player-via its stimulation of inflammation and the nutritional immunity systems, to increase calprotectin and keep the microbiome in a manganese and zinc deficient state. It thus protects its own existence by excluding beneficial lactobacilli and their low pH and oxidative products and contributes to maintaining an environment that is conducive to its own growth and to the introduction of more dangerous BV associated bacteria should sexual or other contact provide a source of these bacteria.

The implication of this understanding is profound and may impact treatment recommendation. Currently, the treatment of an L. iners microbiome is not recommended by the CDC. This recommendation is based on the risk benefit ratio of treatment. The risk of antibiotics enhancing resistant bacterial species vs. the lack of perceived benefit to eliminate L. iners since there has not previously been a causative link between L. iners and BV. In contrast, the findings presented herein show that L. iners is a contributing factor in maintaining an abnormal vaginal environment (dysbiosis) and as such, it would be beneficial to eliminate when present in significant numbers that cause an increase in calprotectin. The simpler treatment described herein consisting of the supplementation of mineral needs for the growth of beneficial lactobacilli would mitigate the treatment risk. This may drive a reassessment of the risk benefit ratios. Treatment of L. iners would remove a source of stimulation of calprotectin production and enhance the restoration of a normal human microbiome with little treatment risk.

Additionally, the inventor has studied L. crispatus and L. iners in in vitro models, but L. jensenii, L. gasseri are also important members of the vaginal microbiome that require further study. L. jensenii, L. gasseri are iron independent, similar to L. crispatus, and are present in low calprotectin environments while absent in BV.

Laboratory findings and analysis, exemplified with the examples presented below, are consistent with the theoretical framework of inflammation, nutritional immunity, and manganese and zinc starvation as being part of the genesis and maintenance of chronic vaginal dysbiosis. The human vaginal microbiome is dominated by lactobacilli, which create an acidic environment thought to protect women against sexually transmitted pathogens and opportunistic infections. Bacterial vaginosis is a dysbiotic condition characterized by a shift from normal vaginal microbes, predominantly lactobacilli in a low pH environment, to anerobic species like Gardnerella vaginalis, Mobiluncus spp. and Atopobium vaginae in an elevated pH environment. The laboratory findings and analysis, together with what is well known about lactobacilli and other bacteria and BV, leads to the theoretical framework summarized above that explains many of the enigmas of BV. While more work needs to be done for an even better understanding, the framework indicates that the methods of treatment of the present invention with manganese and zinc supplementation may be key to re-establishing a vaginal environment friendly to beneficial lactobacilli. Such supplementation especially in conjunction with other vaginal environmental modification such as lactoferrin, oxidants or anti-inflammatories is expected to be compatible with other current proposed individual treatments for BV or its recurrence that have met with limited success, including antibiotics, probiotics, pH modification and carbohydrate supplementation. It also should be useful for the development of therapies for the prophylactic treatment of transitional microbiota. Ultimately, the goal is to always ensure that the vaginal environment is most conductive to the growth of beneficial lactobacilli and additionally not BV associated bacteria. Moreover, the measurement of calprotectin levels may be indicative or even diagnostic of vaginal environments that require Mn supplementation for the treatment or prevention of recurrence of BV.

In one aspect of the invention, disclosed are therapeutic compositions for use in the treatment of a vaginal dysbiotic condition with an elevated level of calprotectin in vaginal fluid, wherein the composition is formulated for intravaginal administration and the composition comprises a dose of essential metal for growth of vaginal flora Lactobacillus, for example at least one D-lactic acid-producing Lactobacillus. The essential metal is in a pharmaceutically acceptable form for intravaginal administration and the essential metal is selected from the group consisting of manganese, zinc and combinations of manganese and zinc.

In some refinements to this aspect, the dose of essential metal in a therapeutic composition can be directly related to elevated calprotectin level in vaginal fluid associated with the dysbiotic condition being treated. In some implementations the dose of essential metal is in an amount to satisfy the following condition (Equation 1):

$$( ( 0.5 \times {Q}_{Zn} ) + {Q}_{Mn} ) \geq ( 0.001 \times K \times {C}_{ECp} )$$

In some other implementations the dose of essential metal is in an amount to satisfy the following condition (Equation 2):

$$( ( 0.5 \times {Q}_{Zn} ) + {Q}_{Mn} ) \geq ( 0.1 \times K )$$

Wherein as used in Equation 1 and Equation 2:
Q_{Zn} is zero or a positive number equal to an amount of zinc in micromoles;
Q_{Mn} is zero or a positive number equal to an amount of manganese in micromoles;
C_{ECp} is a molar concentration of elevated calprotectin in vaginal fluid, in micromoles per liter;
K is a positive number of 1 or greater, optionally an integer of 1 or greater; and
at least one of Q_{Zn} and Q_{Mn} is a positive number.

As may be appreciated, Equation 1 expresses a relationship in which the essential metal dose comprises sufficient manganese and/or zinc that is expected to completely fill calprotectin binding sites for those metals, based on a normal presence of around 1 mL (0.001 L) of vaginal fluid in the vaginal environment. By assuring sufficient metal to fill the corresponding binding capacity of calprotectin in vaginal fluid, at least normally-generated manganese and zinc levels in the vaginal environment should be unaffected by the vaginal calprotectin, to better simulate normal availability of manganese and zinc for use by beneficial Lactobacilli that use manganese instead of iron. These beneficial lactobacilli also produce D-lactic acid. These beneficial Lactobacilli are distinguished from Lactobacillus iners, which uses iron and does not produce D-lactic acid. The relationship expressed by Equation 1 provides significant flexibility to formulate different compositions with different dosages of essential metals to treat different dysbiotic conditions with expected different ranges of elevated calprotectin concentration.

As will be further appreciated, Equation 2 expresses the same relationship as expressed in Equation 1, except assuming treatment of a dysbiotic condition with elevated calprotectin levels assumed at 100 micromoles per liter (100 μM). Data considered by the inventor, although limited, indicates that a number of vaginal dysbiotic conditions involving bacterial vaginosis, or with a transitional microbiome leading to bacterial vaginosis, are expected to have calprotectin levels at around that level or lower, and a composition formulated for that level of calprotectin should have significant utility for treating many dysbiotic conditions, including those that have less than 100 micromoles per liter. For dysbiotic conditions expected to be particularly severe and exceed that level, as will be appreciated the more general relationship of Equation 1 can be used to formulate a therapeutic composition with higher levels of essential metal.

Both Equation 1 and Equation 2 apply a factor of 0.5 to the amount of zinc, in recognition that one mole of calprotectin can bind up to two moles of zinc, whereas no such factor is applied to manganese, because one mole of calprotectin can bind up to one mole of manganese. Also, there is an indication in the literature that calprotectin will bind zinc preferentially to manganese, and so it is expected that even when the therapeutic composition includes only zinc, the zinc will tend to preferentially bind to the calprotectin rather than endogenous manganese binding to the calprotectin, leading to an expectation of manganese availability for use by beneficial Lactobacilli even when a therapeutic composition includes only zinc. In preferred implementations, however, the composition will include some level of manganese.

The K factor in Equations 1 and 2 recognizes that it may often be desirable to increase a dose of essential metal to levels above a stoichiometric minimum based on a variety of considerations. One consideration is that except for a composition formulated for essentially immediate release of the essential metal, release of the essential metal will tend to occur over a period of time and providing an excess of essential metal above a binding capacity of calprotectin provides a source of essential metal at an appropriately elevated level for a meaningful period of time after intravaginal administration of the composition. Another consideration is that vaginal fluid and solutes in the vaginal fluid, such as calprotectin, have some frequency of turnover, and the dose of essential metal should beneficially anticipate such calprotectin turnover. It has been found that calprotectin turns over in vaginal fluid about every 4 hours, or about 6 times per day, and so at a minimum, a therapeutic composition intended for a once per day treatment regimen should preferably be formulated according to either Equation 1 or Equation 2 with a K value of at least 6. However, this is not expected to be a strict requirement, because beneficial Lactobacilli have some capacity to store manganese for some period of time, and so can survive and thrive provided that sufficient manganese is periodically provided in sufficient quantity to cover current needs plus reserve for storage as needed between administrations. Lactobacilli have an ability to store manganese for a significant time of at least about a day and perhaps for up to a week, so that periodic therapeutic administrations should be sufficient even if there is some period of time when vaginal manganese levels drop below the binding capacity of calprotectin for some period of time before the next administration. Additionally, the K factor may be increased even further to better ensure a significant excess of manganese over the calprotectin binding capacity to assure promotion of growth of beneficial Lactobacilli to treat the dysbiotic condition.

As will be appreciated, patients diagnosed with a vaginal dysbiotic condition with elevated calprotectin will exhibit varying levels of elevated calprotectin, and correspondingly varying levels of calprotectin concentration in vaginal fluid. Although Equations 1 and 2 include concentration of calprotectin in vaginal fluid (C_{Ecp}) as a variable, in practice a therapeutic composition may typically be formulated to include a dose of essential metal at a level targeted for treatment of such a patient population with normal patient variation. The essential metal dosage level in a therapeutic composition can be targeted for treatment of the highest concentrations of calprotectin observed to be associated with the vaginal dysbiotic condition, such that the dosage level will be at a larger excess to calprotectin concentration (C_{ECp}) for patients diagnosed with the vaginal dysbiotic condition who have lower concentrations of calprotectin than such maximum observed concentrations. The essential metal dosage level can be varied in different therapeutic compositions targeted to treat different diagnosed vaginal dysbiotic conditions (e.g., pre-bacterial vaginosis vs. bacterial vaginosis). However, given the relatively low toxicity of manganese and zinc, a single therapeutic composition may beneficially be formulated with an essential metal dosage level for treatment of both more severe conditions (e.g., bacterial vaginosis) and less severe conditions (e.g., pre-bacterial vaginosis).

An upper limit on the dose of manganese and zinc is to keep manganese and zinc levels in the vaginal environment below a level of toxicity. Because of the generally low toxicity of manganese and zinc, toxicity concerns are not a significant limitation on the essential metal dose levels. However, from a practical perspective to avoid waste of essential metal, K values are expected to typically be much lower than levels that could be tolerated based on toxicity issues, and accordingly K values are typically expected not to exceed 500.

In addition to the essential metal, the therapeutic composition including essential metal can also include one or more other active ingredients for treatment of the dysbiotic condition, for example to provide synergistic effect with the essential metal. Some other active ingredients that can be included in a therapeutic composition with the essential metal include a probiotic, preferably with one or more D-lactic acid-producing Lactobacillus species, lactoferrin, lipocalin-2, an oxidizing agent, an acidification agent and/or a source of carbohydrate for use by the beneficial, D-lactic acid producing Lactobacilli. As discussed above, treatment with these types of other active ingredients will not be expected to be effective without also counteracting the manganese starvation resulting from elevated calprotectin. Accordingly, with the present invention treatment with such other active ingredients together with or soon following treatment with essential metal to counter calprotectin should provide synergistic benefits not available in the absence of the essential metal treatment.

As will be appreciated, the therapeutic composition may be formulated with a release profile for the intended frequency of administration and the desired duration of elevated essential metal levels to counteract calprotectin. The composition can be formulated to be a fast-release composition, a slow-release composition or an intermediate release composition. With an appropriate release profile and taking into account the ability of beneficial lactobacilli to store manganese for some time, in some implementations it is contemplated that administrations of the therapeutic composition might be spaced at a frequency of about once per week, and possibly even longer. In some preferred implementations, the therapeutic composition will be formulated for a treatment regimen with once daily administrations. The therapeutic composition can include any pharmaceutically acceptable excipients appropriate for intravaginal administration and to provide a desired release profile after intravaginal administration.

The composition can be prepared in any desired form for the intravaginal administration. For example, the composition can be in the form of a vaginal suppository tablet, vaginal ring or other vaginal depot, a vaginal capsule, vaginal cream and a vaginal gel. The composition may be in a discrete form including only a single dose of the essential metal, for example as a vaginal suppository tablet, vaginal capsule or vaginal ring, or may be formulated to provide multiple doses, for example in the form of a cream or gel in a receptacle from which separate aliquots can be removed that each include a single dose for each intravaginal administration. As will be appreciated, formulations such as vaginal gels and creams may alternatively be formulated in separate packaged aliquots each including a single dose of essential metal, if desired.

In another aspect of the invention, disclosed are kits for use in the treatment of a vaginal dysbiotic condition with an elevated level of calprotectin. The kit includes a plurality of administration units each comprising a dose of essential metal for an intravaginal administration. For example the administration units may be in the form of or include the composition of the composition aspect summarized above.

By an administration unit it is meant a formulation amount containing a single dose of essential metal for intravaginal administration, so that the kit includes a plurality of doses of essential metal to be separately administered in separate vaginal administration events. The plurality of administration units may be provided in the kit in discrete separated forms, for example discrete tablets, capsules, vaginal rings or other depot forms or pre-aliquoted gel or cream. Alternatively, the plurality of administration units may be provided in a larger mass designed to be divisible into smaller mass portions each representing a single dose for administration (e.g., a tube or other receptacle with a cream or gel that may be dispensed in separate aliquots each providing a separate administration unit with a single dose). The administrations units may be in any convenient form, for example in any of the example forms listed above for the composition of the previously summarized composition aspect.

The kit may also include a vaginal applicator for use to intravaginally administer an administration unit during a treatment regimen. For example, the vaginal applicator can include a design with a barrel having a distal end to be inserted into the vagina during use and a proximal end to remain outside of the vagina. The barrel can include a lumen between the proximal and distal ends of the barrel, and the applicator can include an interior space to receive and hold an administration unit for insertion into the vaginal. The applicator can include a plunger inserted into the lumen of the barrel from the proximal end and manipulable to be pushed through the lumen to dislodge and push the administration unit into the vagina during intravaginal administration.

The kit may include other active ingredients (e.g., probiotic, lactoferrin, lipocalin-2, oxidizing agent, acidification agent, and/or carbohydrate source). One or more other active ingredient may be included in one or more administration units with the essential metal, or may be provided in one or more separate administration units not including the essential metal. When reference is made herein to an essential metal administration unit, a probiotic administration unit, a lactoferrin administration unit, a lipocalin-2 administration unit, an oxidizing agent administration unit, an acidification agent administration unit or a carbohydrate source administration unit, or the like, the reference is only to an administration unit including the noted active ingredient, but not to the exclusion of also including one or more other active ingredient. For example, if an administration unit includes both a probiotic and lactoferrin, the same administration unit can be referred to as both a probiotic administration unit and a lactoferrin administration unit.

In another aspect of the invention, disclosed are methods of treatment of a person for a vaginal dysbiotic condition with an elevated vaginal calprotectin level. In some implementations of such methods of treatment, essential metal comprising manganese, zinc or manganese and zinc is intravaginally administered to address elevated calprotectin and promote establishment of a healthy microbiome dominated by beneficial Lactobacilli. Essential metal may be administered to increase availability to a level to promote growth in the presence of the calprotectin of one or more D-lactic acid-producing Lactobacillus species and/or to partially or fully counteract manganese starvation stress from elevated calprotectin. In some preferred implementations of methods of treatment, intravaginal administration of the essential metal is sufficient to achieve a metal loading ratio, relative to calprotectin, of 1.0 or larger, wherein the essential metal loading ratio is calculated by the following equation (Equation 3):

$${R}_{EM} = ( ( 0.5 \times {C}_{Zn} ) + {C}_{Mn} ) / {C}_{ECp}$$

wherein,
   R_{EM} is the essential metal loading ratio;
   C_{Zn} is zero or a positive number equal to a concentration in micromoles per liter of zinc in the vaginal fluid; and
   C_{Mn} is zero or a positive number equal to a concentration in micromoles per liter of manganese in the vaginal fluid;
   C_{ECp} is the analytically-determined elevated calprotectin concentration in micromoles per liter; and
   at least one of C_{Zn} and C_{Mn} is a positive number.

Similar to the K factor with Equation 2 and Equation 3, an R_{EM} value of 1.0 represents a minimum condition of attaining a stoichiometric concentration in the vaginal fluid to fully counteract the binding capacity of calprotectin. As will be appreciated, it will be preferred to have the value of R_{EM} exceed 1.0, and more preferably to significantly exceed 1.0, and for a meaningful duration to permit beneficial Lactobacilli to acquire, use and store manganese to promote desired growth of the beneficial Lactobacilli. As the beneficial Lactobacilli have an ability to store manganese for a significant duration (for at least a day and possibly as long as a week), it is not necessary that the loading ratio be maintained at all times between intravaginal administration events of the essential metal during a treatment regimen.

In addition to periodic intravaginal administrations of essential metal, a treatment regimen may also include administration of one or more other active ingredients, for example one or more of a probiotic, lactoferrin, lipocalin-2, an oxidizing agent, an acidification agent and/or a carbohydrate source. Such other active ingredients can be administered together with the essential metal, for example in a single administration unit with the essential metal, and/or one or more or such other active ingredients may be administered separately from the essential metal, for example in one or more separate administration units not including essential metal. Such another active ingredient may be administered contemporaneously with administration of essential metal, or may be administered at a different time during a treatment regimen protocol. With the exception of the probiotic, which can be effectively administered orally or intravaginally, these other active ingredients should be administered intravaginally. Even for the probiotic, intravaginal administration is a preferred route.

As a treatment regimen proceeds and the dysbiotic condition recedes with establishment of a microbiome dominated by beneficial Lactobacilli, inflammation should recede and calprotectin levels decrease. Monitoring of calprotectin levels at selected times after commencement of an essential metal treatment regimen can provide information on progression of treatment, including when to discontinue treatment. Such monitoring of calprotectin can be supplemented by monitoring one or more other indications of microbiome health, for example, levels of D-lactate, L-Lactate, acetate and/or ORP in vaginal fluid. Higher levels of D-lactate is indicative of the presence of thriving beneficial Lactobacilli, higher levels of L-lactate, especially relative to D-lactate, is indicative of presence of the undesirable presence of Lactobacillus iners, and higher levels of acetate is indicative of presence of undesirable presence of bacteria associated with bacterial vaginosis. Alternatively, metabolomic parameters consisting mainly of amino acids, decarboxylated amino acids and amines may be used to assist in the tracking of BV. Calprotectin, however, occupies a prime position, as it is the final mechanism in the body's mechanism to suppress lactobacilli growth via manganese and zinc binding.

In another aspect of the invention, disclosed are methods of analyzing vaginal microbiota condition. The method comprises assaying vagina fluid to determine a level of calprotectin in the vaginal fluid. As summarized above, and elevated level of vaginal calprotectin is an indicator for vaginal dysbiotic conditions, for example associated with bacterial vaginosis or a transitional condition preliminary to bacterial vaginosis (pre-bacterial vaginosis). The absence of elevated calprotectin will tend to indicate an absence of a dysbiotic condition in which the beneficial Lactobacillus have been or are being replaced with less desirable bacterial species such as L. iners or bacteria associated with bacterial vaginosis. An elevated calprotectin level may indicate the presence of such a dysbiotic condition.

To differentiate possible conditions and level of severity, the assay for calprotectin may be combined with an assay for one or more other analyte in addition to calprotectin, which may be performed on the same vaginal fluid sample as used to assay for calprotectin, or a different vaginal fluid sample, preferably obtained near in time to the vaginal fluid sample used to assess calprotectin level. Such an other analyte might include, for example, D-lactate, with or without assaying also for L-lactate. A low or absent level of D-lactate is indicative of a dysbiotic condition with diminished presence of beneficial Lactobacilli in the microbiome. A relatively high level of L-lactate relative to D-Lactate is indicative of a significant presence of Lactobacillus iners. Another possible useful analyte for distinguishing whether a condition is dysbiotic and the nature of a dysbiotic condition is acetate. An unusually high concentration of lactate is indicative of the presence of bacteria associated with bacterial vaginalis. In combination with calprotectin assay information, assay information on one or more of these additional analytes provides additional information to distinguish elevated calprotectin associated with inflammation of a dysbiotic condition from calprotectin that may be associated with a different type of condition, and to help identify the type of dysbiotic condition (e.g., bacterial vaginosis vs. pre-bacterial vaginosis) and relative severity. Furthermore, the analysis of these compounds and metabolomic analysis, as above, may help to differentiate BV form other gynecological conditions that may be shown to share an elevated or spiked level of calprotectin, during their clinical course, but in which elevated calprotectin levels have not yet adversely impacted the microbiome in a significant way.

The method may also include performing a microbiota assay, for example by PCR, to determine vaginal microbiome composition. The microbiota assay may include assaying for a first bacteria comprising one or more D-lactic acid-producing (beneficial) Lactobacillus species and a second bacteria associated with a dysbiotic condition, for example L. iners and/or one or more bacteria associated with bacterial vaginosis.

An important consideration for effective analysis is repeatability and control on vaginal fluid samples collected and preserved prior to analysis. A normal amount of total vaginal fluid on all the mucosal surfaces of the vagina is estimated to be on the order of 1 mL, and accordingly vaginal fluid samples tend to be very small volumes. Although vaginal fluid samples in a range of up to 100 microliters might be obtainable via swabbing from some patients, more typical is 20 to 30 microliters, and from some patients it is not possible to obtain samples even of that size. This is a different situation than many biological fluid tests such as blood, where repeatable acquisition of relatively large fluid samples is routine. It has been identified in connection with the present invention that for vaginal fluid samples used for the method of analyzing microbiota condition, it is important to plan on collecting a calibrated amount of a very small volume and to take care to collect and preserve the vaginal fluid samples in a neat form prior to analysis in conditions to maintain sample integrity. Such very small, calibrated volumes of neat vaginal fluid are particularly susceptible to loss of fluid volume through evaporation of liquid, which can significantly alter sample volume and accordingly may result in significant inaccuracy of reported concentrations or calprotectin and other analytes. By a neat vaginal fluid sample, or neat form, it is meant a sample of vaginal fluid as taken from a patient, and without compositional modification through dilution or addition of reagents. Collecting such a neat fluid sample may be accomplished for example by obtaining a swab sample of vaginal fluid and then squeezing the neat vaginal fluid out of the absorbent material of the swab until the calibrated volume is obtained or centrifuging the swab to separate neat vaginal fluid components from the absorbent material of the swab until the calibrated volume is obtained. The resulting calibrated volume of the vaginal fluid sample is then either used on-site for analysis without significant delay or is quickly put into a small container that has a maximum fluid containment size close to that of the small, calibrated volume of the neat vaginal fluid sample, and with little air head-space, to avoid significant evaporative loss of sample volume to vapor headspace that would otherwise be present in larger containers that may be more generally used for other purposes. The container may be sealed and stored and transported while preserved in the sealed container to preserve the integrity of the neat vaginal fluid sample until opened in connection with performing a method of analyzing for microbiota condition according to this aspect. As a further enhancement, it would be desirable to have a specialized sample collection tool or combination to facilitate easy and repeatable collection and provision of a neat vaginal fluid sample of the desired small, calibrated volume for standardized analysis.

Another important consideration in assaying vaginal fluid samples to identify elevated calprotectin levels, especially for use to diagnose a vaginal dysbiotic condition, is good repeatability of assay results, leading to better consistency for accurate proper diagnosis. Any assay technique that provides a repeatably reliable indication of elevated calprotectin level that correlates with the occurrence of a vaginal dysbiotic condition can provide useful information to aid diagnosis of the vaginal dysbiotic condition. Such an assay technique can be targeted toward determination of a close estimate of total amount or total concentration of calprotectin in a vaginal fluid sample, or to determine calprotectin occurrence in only a specific portion or portions of a vaginal fluid sample that provide useful indications of elevated calprotectin levels that reasonably correlate with a particular vaginal dysbiotic condition. In the examples presented in the EXAMPLES section below, vaginal fluid samples obtained in a swab were centrifuged, during which the vaginal fluid samples are separated into a supernatant liquid fraction and denser pellet containing a separated solids fraction, and the liquid fraction was analyzed by ELISA for calprotectin level, and the measured calprotectin levels provide reasonable correlation with presence or absence of bacterial vaginosis.

However, it has also been determined that measured levels of calprotectin from such a separated liquid fraction can vary significantly between samples and that improved repeatability and better correlation generally with bacterial vaginosis can be obtained by further analysis for additional calprotectin associated with the separated solids fraction. In that regard, it has been found that significant calprotectin can be associated with such separated solids fraction, and that accounting for that additional calprotectin can both provide better repeatability of calprotectin assay results and a closer approximation of actual calprotectin concentration in a vaginal fluid sample. It has been found that some calprotectin appears to be adhered to the separated solids fraction and can be recovered for analysis by washing the solids fraction with a buffer solution at a higher pH than the normal acidic pH of vaginal fluid, and that buffer solution at about pH 7.4 appears to work well for dissolving that calprotectin, and the resulting wash solution with the additional dissolved calprotectin can be analyzed, for example by ELISA or other analysis techniques, to account for that additional calprotectin in the vaginal fluid sample. Current experience indicates that often about 30% to 70% of total calprotectin in a vaginal fluid sample can be associated in this manner with such a separated solids fraction and recovered for assay by such a wash procedure, and even amounts greater than 70% have been observed in some clinical conditions. Because this calprotectin is recoverable by a simple dissolution in a wash solution, it is identified as extracellular calprotectin associated with the separated solids fraction. The calprotectin recovered in the liquid fraction is also referred to as extracellular calprotectin. Adding together the measured calprotectin recovered in the separated liquid fraction with the calprotectin recovered in the pellet wash liquid has been found to provide a better correlation to occurrence of bacterial vaginosis, and also to provide a better basis for estimating actual concentration of calprotectin in the vaginal fluid sample.

It has also been determined that there is some calprotectin in vaginal fluid samples that can be contained in cellular components of such a separated solids fraction. This calprotectin is referred to as intracellular calprotectin. Following the noted pellet wash procedure to remove extracellular calprotectin adhered to the solids fraction, such intracellular calprotectin can then be recovered by using a detergent-like solution to lyse cells in the residual solids fraction and dissolve the intracellular calprotectin, and the resulting solution can then be analyzed for calprotectin content, which can also be performed for example by ELISA or other analysis techniques. Such intracellular calprotectin appears to not significantly affect correlation to occurrence of bacterial vaginosis. Furthermore, failure to wash the pellet could result in attributing extracellular calprotectin to intracellular calprotectin.

For enhanced effective utilization of calprotectin assay results, it is important to provide normalization of assay results in a way that reduces scatter and improves evaluations for correlation of vaginal calprotectin levels across patient populations. That can be accomplished through expressing assay results as a concentration value for assayed calprotectin quantity relative to a unit quantity value of the assayed vaginal fluid sample or of a measured portion of the vaginal fluid sample. The unit quantity value can be on any basis for any convenient units for quantification, for example on a volume basis (e.g. per liter) or mass or weight basis (e.g., per gram), and likewise the quantity of calprotectin can be expressed in any convenient units (e.g, volume, weight or mass or molar units). However, to provide such a concentration value some identified part or parts of the vaginal fluid sample must be measured with reasonable accuracy. This presents a problem, for example, in attempting to use samples of vaginal material taken by sampling procedures such as a cervicovaginal lavage, where the vaginal environment is flushed with an irrigation fluid to collect a sample of vaginal material. However, such techniques are not amenable to accurate quantification of the amount of vaginal material collected, presenting a significant problem for normalization of assay results expressed as useful concentration values for evaluation and correlation across patient populations. It has been found, however, that useful concentration values can be calculated even in these types of situations by centrifuging the sample to obtain a separated solids fraction (centrifuged pellet phase), which is known to be sourced from the vaginal environment, and to use the measured mass or volume (preferably mass) of that solids fraction as the basis for a concentration determination (e.g., grams or moles of calprotectin per gram or liter of pellet material). However, when a neat vaginal fluid sample is obtained (not a diluted, irrigated sample such as in a lavage), then a measured quantity of the whole vaginal fluid sample assayed or a measured portion of the sample (e.g., measured volume of mass of separated liquid fraction or measured volume or mass of separated solids fraction, or a combined value for separately measured liquid and solids fractions) are quantified. Moreover, further granularity and usefulness of results are available when neat vaginal fluid samples are collected and assays are performed on different portions of the neat sample, as described above, to identify and normalize as concentration values the calprotectin occurrences in different identified portions of the vaginal fluid sample (e.g., extracellular calprotectin in neat separated liquid fraction, extracellular calprotectin washed from separated solids fraction or intracellular calprotectin extracted from cellular content of separated solids fraction).

In another aspect of the invention, disclosed are methods of diagnosing a vaginal dysbiotic condition, for example associated with bacterial vaginosis or pre-bacterial vaginosis. The methods include evaluating an assayed vaginal calprotectin level in vaginal fluid of a person, which may beneficially be provided by a method of analyzing for microbiota condition as provide by the previously summarized aspect. The presence of a vaginal dysbiotic condition is diagnoses based at least in part on determining that there is an elevated level of calprotectin. The method may also include evaluating an elevated level of calprotectin with one or more other positive indications of the vaginal bacterial dysbiosis. Such other positive indications may, for example, be other indications resulting from a method of evaluating microbiota of the previously summarized aspect, and/or one or more traditional patient symptoms indicative of the dysbiotic condition.

In yet another aspect of the invention, disclosed are methods of discontinuing treatment of a vaginal dysbiotic condition, which may include a treatment regimen with intravaginal administration of essential metal as disclosed for the aspect first summarized above, or may include a treatment regimen not involving a method of treatment of the present invention. In any event, the method includes monitoring at least a level of calprotectin, and optionally one or more other indications of microbiota condition, such as those disclosed in connection with the aspect of this disclosure concerning methods of analyzing microbiota. Treatment may be discontinued after the calprotectin level in vaginal fluid has dropped below a threshold level indicative of successful treatment of the dysbiotic condition, alone or in combination with attaining other indications of successful treatment.

Numerous additional features and advantages of the present disclosure will become apparent to those skilled in the art upon consideration of the embodiment descriptions provided hereinbelow.

### BRIEF DESCRIPTION OF THE DRAWINGS

FIG. 1 depicts concentrations of Mn and Zn in vaginal fluid compared to metal binding sites in calprotectin in vaginal fluid FIG. 2 depicts Mn and CP concentrations and corresponding bacteria found in vaginal fluid samples. The bacteria further categorized as normal flora being dominated by beneficial or good lactobacilli or dysbiotic flora is defined as Gardnerella vaginalis and/or the transitional species L iners.

FIG. 3 depicts growth curves for L. crispatus and L iners in aerobic, microaerophilic and anerobic conditions.

FIG. 4 depicts levels of calprotectin in healthy women and women with bacterial vaginosis, n=16 per group, line indicates median value (22 g/mL vs 223 μg/mL for healthy vs BV).

FIG. 5 depicts concentrations of manganese and zinc in vaginal fluid from women with unknown BV status and concentrations of calprotectin in vaginal fluid from women with healthy (L. crispatus dominated), L iners-dominated or BV vaginal microbiomes.

FIG. 6 Panel A depicts predicted concentrations of manganese in vaginal fluid after administration of a 50 μg dose of Mn in slow release formulation. Dotted lines show min and max concentrations of endogenous Mn from n=20 women. Dashed lines show amount of Mn bound by calprotectin in BV (n=24).

FIG. 6 Panel B depicts predicted concentrations of manganese vaginal fluid after administration of a 250 μg dose of Zn in a slow release formulation. Dotted lines show min and max concentrations of endogenous Zn from n=20 women. Dashed lines show amount of Mn bound by calprotectin in BV (n=24).

FIG. 7 depicts the relative distribution of assayed calprotectin (CP) fractions associated with different portions of vaginal fluid samples recovered from vaginal swabs with centrifugation. The fractions of calprotectin include extracellular calprotectin in a separated liquid fraction following the centrifugation, extracellular calprotectin recovered from a separated solids fraction following the centrifugation in a pellet wash solution and intracellular calprotectin extracted from lysed cells of the separated solids fraction following the pellet wash procedure. The brackets represent paired samples from the same patient.

### DETAILED DESCRIPTION

The methods of treatment, and related compositions and kits, are generally directed to treatment of vaginal dysbiotic conditions accompanied by an elevated level of calprotectin. Such vaginal dysbiotic conditions may be bacterial vaginosis or may be of a transitional nature preliminary to full development of bacterial vaginosis. In that sense, various methods of treatment disclosed herein may be prophylactic for development of bacterial vaginosis when a treatable preliminary transitional dysbiotic condition is diagnosed before development of bacterial vaginosis.

The discussion herein is presented primarily with reference to treatment of bacterial vaginosis, but the same principles apply to treatment of dysbiotic conditions concerning pre-bacterial vaginosis, although as will be appreciated, the level of treatment provided may be scaled and tailored to the particular severity of the dysbiotic condition at issue in a particular circumstance.

The concept of nutritional immunity was recently further expanded, when it was discovered that manganese and zinc are part of this system, as they are sequestered by the protein calprotectin. Sequestration of these metals is accomplished by these protein binding sites that are highly specific for certain metals and ionic states. This is unlike other chelators, such as EDTA that non-selectively binds many multivalent metals. It must be emphasized that while nutritional immunity does not target a specific species, it inevitably affects some bacterial species more than others, since comparatively, bacterial baseline requirements and internal bacterial reserve capacity for these metals vary. Furthermore, some bacteria have developed alternative methods which they up-regulate when iron deprived, in order to source these critical minerals despite the body's attempts at sequestration. Several known mechanisms include 1) specialized bacterial transport mechanisms to source mineral needs from low concentration environments, 2) specialized bacterial binding proteins that directly extract iron from chelation proteins and 3) siderophores produced by bacteria and then secreted into the environment that scavenge iron, using their high affinity receptors. The siderophore-iron complexes are then collected by the bacteria. Most often, this tug-of-war is not won decisively by either side, but a given bacteria's ability to work-around the body's defenses determines the ability to live commensally within the body. BV is an example of a significant and most often long term invasion of bacteria that are able to live within the body's ecosystem while at the same time cause significant symptoms and risks.

Conceptually, nutritional immunity is designed to starve bacteria of their critical nutrients. Enrichment culture in the laboratory, on the other hand aims to do the opposite. It enhances the environment for a given bacterial species, taking into account its specific nutritional needs. In a biological system, these two concepts may coexist and may be individually manipulated to enhance selective bacterial growth of one species over another. However, being able to do so in vivo depends on 1) there being significant enough differences in nutritional needs of the respective species 2) a precise understanding of existing nutritional conditions and 3) an ability to modify and maintain control over the environment until the new environment and resulting microbiome has developed and is intrinsically stable. The methods of treatment of the invention have an objective of meeting these criteria in BV.

In an embodiment a new therapy may suppress pathological BV bacteria and L. iners, a transitional bacteria. In an embodiment the therapy is directed to restoring beneficial bacteria associated with a normal microbiome. In another embodiment the therapy can be utilized as a follow up therapy, for example after a course of antibiotics to make the microbiome healthier and diminish the high recurrence of BV.

The invention provides methods of treatment of BV by vaginal supplementation of essential metals, optionally in combination with other agents such as minerals and carbohydrates, to encourage the growth of beneficial lactobacilli to the detriment of BV bacteria and Lactobacillus iners. In an embodiment a method of treating and preventing bacterial vaginosis recurrence in women comprises vaginally administering a therapeutic composition capable of encouraging growth of D-lactic acid producing bacteria and selectively reducing growth of bacteria associated with bacterial vaginosis.

In another embodiment treatment with a method of the invention includes iron sequestration, together with the vaginal supplementation of essential metals, to encourage the growth of beneficial lactobacilli to the detriment of BV bacteria and Lactobacillus iners. In an embodiment a method of treating and preventing bacterial vaginosis recurrence in women comprises vaginally administering a therapeutic composition capable of sequestering iron and selectively reducing growth of bacteria associated with bacterial vaginosis.

In another embodiment treatment with a method of the invention can be used in conjunction with standard of care therapy, either before, during or after antibiotic treatment to help restore a more normal microbiome. Those therapeutic approaches that leave L. iners in place after the acute therapy for BV are likely to fail in the long term since when L. iners significantly persists, it provides an opportunity for BV bacteria to enter the vaginal microbiome and flourish.

Probiotics to replace L. iners with other Lactobacillus species is another interesting theory that has undergone much study in recent years with mixed results. The probiotic approach is thought to encourage growth or seed good bacteria but it does not complete the treatment algorithm proposed of changing the environment such that transitional bacterial like L iners are at a disadvantage. This may explain the limited effectiveness of probiotics. The methods of treatment of the invention, if combined with probiotic therapy, may significantly increase the effectiveness of such therapy.

Beneficial D-lactate producing lactobacilli require high manganese levels to grow optimally as shown by Macleod (1947). These have evolved in a highly unusual way to use manganese instead of iron for redox reactions. Like iron, manganese can transition between its Mn 2+ and Mn 3+/4+ states and provides the mechanism for electron transport. As can be expected for these non-iron-dependent bacteria, a much higher nutritional level of manganese is required if lactobacilli are to thrive. Lactobacillus selective (MRS) media designed by DeMan Rogossa and Sharpe (1960) have high levels of manganese and low levels of iron relative to other, more standard bacterial culture media. This specialized media permit lactobacilli to grow, yet do not provide sufficient nutrition for other bacteria to grow. This illustrates that at least in vitro, nutritional methods in a controlled environment can enhance the specific growth of lactobacilli over other bacteria that have sufficiently different metabolic and nutrient needs.

Falsen (1999) was the first to describe L. iners; over 100 years after vaginal lactobacilli were described by Doderlin. Unlike other lactobacilli, this unusual member of the species has an atypical microscopic appearance, has a requirement for iron, does not grow on MRS lactobacillus selective media, does not metabolize lactose (milk sugar) and produces L-lactate instead of D-lactate. It also does not require specific manganese supplementation in its growth media. These characteristics were unusual and noteworthy for a lactobacillus but are essentially normal for most if not all other BV bacteria. L. iners can source its iron requirements either directly or in symbiosis with BV bacteria such as G. vaginalis that lyse red blood cells during menses.

A treatment algorithm has been developed that accomplishes this, by combining data with an in-depth knowledge of the genetic, metabolic, nutritional, and evolutionary aspects of the critical bacterial species in both the normal and BV microbiota. The algorithm capitalizes on the highly divergent metabolic and nutritional needs of the respective bacterial groups within the context of knowledge of the existing vaginal environment. It is theorized that once the vaginal environment is enhanced and a normal microbiome is restored, the microbiome will be robust and self-sustaining, as it is in most women without BV. Reinfection may recur but it will be less frequent if the microbiome is fully restored to normal with an abundance of functioning protective lactobacilli. This theory has been developed and augmented by data collection. Specialized techniques have been developed to collect and analyze tiny amounts of pure vaginal fluid. Multiple specimens of vaginal fluid from normal woman and those with BV are collected and analyzed in order to understand the normal vaginal ecosystem and microbiome as well as the disease state, identifying and quantifying important parameters for each of them. These assays determined baseline levels of iron, manganese, zinc and the protein calprotectin in vaginal fluid (a protein which binds manganese and zinc). A treatment plan was developed based on assembling the data points into a unified understanding and novel approach.

This original data shows that manganese levels in the vaginal fluid of both normal women and those with BV is approximately 1 percent of the manganese concentration in optimized lactobacillus culture (MRS) media. The inventor's assessment of this deficiency is based on the empiric, required levels of manganese to optimize in-vitro laboratory growth. These levels of manganese in vaginal fluid are additionally compromised since they are bound tightly by calprotectin, which are found to be greatly elevated in BV. On average, calprotectin is elevated 10 times relative to normal patients without BV. Calculations show that the elevated calprotectin levels in vaginal fluid seen in BV are sufficient to completely bind all of the manganese contained in the fluid. Thus manganese supplementation may be critical in treating BV and restoring of the normal vaginal microbiome.

Zinc levels in vaginal fluid of BV have also been measured. Recommended zinc levels for bacterial growth are not readily available as zinc toxicity is low and the capacity of bacteria to store zinc is high, thus contributing to a very wide range of acceptable zinc levels that will support bacterial growth. Analysis of the levels of calprotectin, its binding capacity and the levels of manganese and zinc show that in BV, calprotectin is sufficient to bind all available zinc and manganese contained in vaginal fluid. Furthermore, in an L. iners based microbiome that has not progressed to full blown BV, a significant increase in calprotectin is also seen. Thus, when an altered microbiome takes hold, the body appears to be an active conspirator in discouraging L. Crispatus-type re-growth. It does so by binding manganese and zinc, which as described above, affects L. Crispatus-type bacteria to a much greater extent than BV bacteria, and transitional bacteria like L. Iners. To determine the dose needed to obtain a given intravaginal concentration of a given therapeutic, extensive pharmacokinetic studies are performed on lactoferrin and an inert undigested marker (mannitol). This data has established both concentration and duration of a given vaginal dose and the route of elimination.

In summary, the aforementioned data illustrates several keys concepts: 1) L. iners is quite different from L. crispatus type bacteria in its metabolic and nutritional needs and is most similar to BV bacteria except in its antibiotic sensitivity, 2) in the vaginal environment, the body's nutritional immunity mechanism is quite active especially in BV with elevated inflammatory markers, lactoferrin levels and highly elevated calprotectin levels, and 3) the data provides strong clues and a consistent theory as to why L. iners and BV bacteria can out-compete L. crispatus, from a nutritional standpoint, and 4) provides insight to possible treatment algorithms. The details of the nutritional needs of BV bacteria and L. iners are highly significant if we are to modify the vaginal environment in line with specific nutritional parameters.

Once established after displacing the normal vaginal microbiome, L iners and other pathological bacteria are sustained as a viable microbiome since 1) they are able to source iron during menses and have highly developed mechanisms to source iron during other periods of the monthly cycle 2) they are able to source their requirements for trace amounts of Mn even from the low levels present in vaginal fluid, and 3) they can obtain sufficient levels of zinc with the aid of zinc acquisition pathways and 4) as a group, they are able to metabolize proteins in addition to glucose, and 5) BV bacteria and L. iners can be part of the same microbiome and share any of the above resources. One therapeutic nutritional impact approach may be to strengthen the body's attempt at nutritional immunity via human lactoferrin, by augmenting it with an excess of iron depleted bovine lactoferrin and lipocalin-2 (LCN2) against siderophores. Siderophores are compounds that are created by bacteria to bind iron, they are secreted into the environment, and reabsorbed after they have bound iron. They have an extremely high affinity for iron and can source iron even from lactoferrin. G. vaginalis has been shown to make these compounds under conditions of iron starvation. To counter bacterial siderophores, and as part of the cat-and-mouse struggles for iron, the body manufactures “anti-siderophore” compounds that bind siderophores, even those loaded with iron and thus prevent uptake by bacteria. The protein described is lipocalin-2 (LCN2), a 25 kDa compound that has specific high affinity for the “catecholate-type” siderophores that G. vaginalis produces. In contradistinction, L. crispatus-type bacteria are relatively disadvantaged in the identical environment since 1) their manganese requirements are very significant and their requirements are difficult to source in the manganese-poor vaginal environment, 2) they lack a zinc transport system to extract zinc from the low-zinc vaginal environment, and 3) they can only metabolize glycogen that has been predigested by a vaginal amylase enzyme that they do not even themselves possess, 4) the relatively easy availability of iron during menses is of no benefit to them since they do not use iron in their metabolic reactions or enzymes. Their nutrition may be positively augmented by providing for example, manganese, zinc and lactose.

The vaginal microbiome begins development before puberty and prior to significant competition from bacteria who can source iron from menses. The inventor hypothesizes that over time, a mature and stable L. crispatus-like microbiome is formed with adequate accumulated stores of manganese and zinc. Once established, well-recognized metabolic products of low-pH lactate and hydrogen peroxide provide a barrier to the colonization of other bacteria. If, however, a significant colonization with L. iners occurs, it will be entrenched in a vaginal environment that is well suited for its continued viability and has little reason to yield to recolonization by L. crispatus-type bacteria. This is especially true with the added deleterious effect of inflammation in BV where the body actively sequesters, via elevated calprotectin, the critical nutrients of manganese and zinc, that impact L. crispatus-like bacteria to a greater extent than L. iners. The precise levels of manganese, zinc and calprotectin identified in the vaginal fluid of normal and disease states, suggests a mechanism for BV and recurrence that has not been previously described. It attributes to and defines the underlying fundamental characteristics of the of a vaginal ecosystem which make it best suited at that point in time for the growth of transitional and BV microbiomes to the disadvantage of L. crispatus-type bacteria. As such, the treatment algorithm presented herein aims to change the vaginal environment in as many ways as possible to limit or reverse this preference.

In one embodiment our algorithm is focused on manganese and zinc individually and in combination; precisely the minerals and metals that body attempts most to sequester. Supplementing manganese at levels that overwhelm the body's ability to sequester manganese via calprotectin and to levels that are similar in magnitude to what has been found to work best in laboratory selective media for Lactobacillus species. As such, any advantage that iron-based bacteria have in the plentiful vaginal environment over those that use manganese is lost. Zinc is augmented to remove the advantage that some BV bacteria have, including L. iners, by their ability to source critically necessary zinc in zinc-poor environments, such as the vagina. Zinc is also added to fill calprotectin's binding sites as the same sites that bind manganese also bind zinc.

In brief, the levels of supplementation for manganese and zinc are set to exceed the highest possible level of calprotectin sequestration based on our original collected data regarding calprotectin elevation in vaginal fluid and the levels that are typical media concentrations that have been developed to grow these bacteria optimally in-vitro. These limits should not be viewed as limiting as during further study in larger cohorts, since these values may vary especially in biological systems where other environmental factors may impact a bacteria's access to the dosed trace metals.

In another embodiment the therapeutic composition comprises manganese as an active pharmaceutical ingredient formulated in the therapeutic composition, for the treatment of bacterial vaginosis either alone or in combination with other materials. Manganese is a trace element/metal which is an essential nutrient and can be administered as a therapeutic composition of the present invention to enhance the growth of lactobacillus dominated vaginal microbiome. In general, the lactobacillus family utilize manganese instead of iron and, therefore, their requirements for manganese are much higher than those of other species. The inventor proposes manganese supplementation to encourage the selective bacterial growth of Lactobacilli in patients with bacterial vaginosis, as a primary treatment or as an accessory treatment for bacterial vaginosis at levels and duration sufficient to support robust growth of beneficial lactobacilli. This will enhance the ability of lactobacilli to compete with and ultimately displace those bacteria that are dependent on iron, as all BV associated bacteria are, including the transitional bacteria L. iners. As discussed above, without intervention, these BV associated bacteria have a built-in advantage over beneficial lactobacilli since they can obtain iron during menses and many have otherwise developed iron binding receptor proteins or specialized iron scavenging proteins (siderophores) to satisfy their iron needs. The inventor theorizes a therapy that enhances the growth of L. crispatus type bacteria without similarly enhancing the growth the transitional bacterial L. iners is particularly desirable. Manganese supplementation will provide a necessary nutrient that is currently deficient in those that have developed BV, even after apparent clinical symptomatic recovery.

In an embodiment manganese is added as an active pharmaceutical ingredient to a therapeutic composition also containing lactoferrin, and optionally one or more excipients such as a binder, a filler and a gelling agent. This approach is based on utilizing the iron-sequestering capacity of lactoferrin to modify the vaginal microbiome and restore the normal resident microbiota. In newborns, lactoferrin from mother's milk promotes a predominantly lactobacillus and bifidobacterial gut microbiome. As in the intestinal microbiota, lactobacilli also comprise a healthy vaginal microbiota. The iron-sequestration mechanism of lactoferrin action depends on the fact that virtually all bacteria need iron. Lactobacilli are an exception and are not suppressed because they do not require iron, using manganese instead. Measurements of manganese levels in vaginal fluid show that they are low, relative to optimal levels for lactobacillus growth contained in optimized laboratory culture media, further diminishing the availability of manganese to bacteria. Furthermore, original research has identified significant levels of calprotectin in vaginal fluid, a specialized protein that is known to bind manganese with a high affinity and makes it unavailable for use by bacteria. The necessity of robust levels of available manganese is further highlight by the fact that lactobacillus selective media such as DeMan, Rogosa and Sharpe media, contain no supplemental iron but do contain an excess of manganese as compared to typical human physiological levels and other bacterial growth media. Manganese supplementation to encourage the selective bacterial growth of Lactobacilli in patients with bacterial vaginosis, as a primary treatment or as an accessory treatment with lactoferrin for bacterial vaginosis is a novel idea that has not been previously described.

The therapeutic compositions of the invention comprises at least one essential metal for growth of Lactobacillus as an active pharmaceutical ingredient, and optionally the composition also comprises one or more excipients, for example a binder and/or a filler, formulated for intravaginal administration for the treatment of bacterial vaginosis. Essential metal in the composition includes one or both of manganese and zinc. Additionally, the composition may include one or more other trace metals, for example copper. Non-limiting examples of other possible therapeutic components in the composition for growth of Lactobacillus comprise bovine lactoferrin, lipocalin-2 (LCN2), and copper. Non-limiting examples of beneficial, D-lactate producing Lactobacilli include: L. crispatus, L. jensenii, L. johnsonii, L. gasseri, L. helveticus, L. rhamnosus, L. reuteri, L. casei, and L. plantarum.

In some embodiments, the therapeutic composition may contain one or more other active pharmaceutical ingredients. for example proteins, peptides, minerals, metals, antibiotics, anti-inflammatories, cytokines, hormones, anesthetics, analgesics, probiotics and fragments thereof.

In some embodiments, a therapeutic composition further comprises a probiotic, preferably comprising at least one D-lactic acid-producing Lactobacillus species.

In some embodiments, a therapeutic composition further comprises lactoferrin, which may be a native lactoferrin or an iron-depleted lactoferrin, and is preferably bovine lactoferrin.

In some embodiments, therapeutic composition further comprises lipocalin-2.

In some embodiments, a therapeutic composition further comprises an acidification agent to help lower the pH of the vaginal environment wherein the agent is selected from the group consisting of boric acid, lactic acid and a lactate.

In some embodiments, a therapeutic composition further comprises an oxidizing agent, for example selected from the group consisting of hydrogen peroxide and urea peroxide.

In some embodiments, a therapeutic composition further comprises a carbohydrate source for the beneficial lactobacilli, for example selected from the group consisting of lactose and sucrose.

As an alternative to including one or more additional agents, such as a probiotic, lactoferrin, lipocalin-2, oxidizing agent, acidification agent or carbohydrate source in a combined composition with manganese and/or zinc, such an additional agent for a therapeutic treatment may be provided in a separate composition that is administered contemporaneously with administration of the essential metal or not contemporaneously with administration of the essential metal but administered at appropriate times during a treatment regimen to effectively supplement action of the essential metal. Similarly, although it is generally preferred, at least for convenience, to provide manganese and zinc together in a single administration composition when a method included treatment with both manganese and zinc, it is also possible to vary the treatment to administer manganese and zinc in separate compositions, which separate compositions may be intravaginally administered contemporaneously or may be administered at different times, for example alternating administrations of manganese and zinc.

The therapeutic composition delivered intravaginally, containing the essential metal, can be formulated as immediate release or a slow release composition, or with an intermediate release profile. In that regard, the therapeutic effect may be due to the direct action of the pharmaceutical ingredient of the composition or may be due to one or more other materials synergistically interacting with the pharmaceutical ingredient. In some embodiments, the present invention provides a slow release therapeutic composition comprising an active pharmaceutical ingredient, a binder, a filler and a gelling agent that, as formulated in the therapeutic composition delivered intravaginally for the treatment of bacterial vaginosis. By treatment of bacterial vaginosis, it is meant that the therapeutic composition is effective to prevent or reduce the incidence, severity and/or duration of the infection. The therapeutic composition may also prevent reoccurrence of the infection.

The therapeutic composition can be in any convenient physical dosage form, with one preferred form being a pessary. For example, when treating bacterial vaginosis, the therapeutic composition is preferably in the form of a tablet which can be introduced intravaginally. Nonlimiting examples or other dosage forms include a vaginal ring or other vaginal depot, a vaginal capsule, vaginal cream or a vaginal gel.

In another embodiment, a tablet may consist of two or more distinct layers, with each layer having its own blend of excipients and therapeutics and each having its own pharmaco-kinetic dissolution profile depending on the dissolution needs of the particular therapeutic.

In another embodiment, a tablet may consist of two or more distinct layers, with each layer having its own blend of excipients and therapeutics and each having its own pharmaco-kinetic dissolution profile depending on the dissolution needs of the particular therapeutic. A combination of a quicker release layer and a slower release layer, would provide in total prolonged dissolution profiles and prolonged therapeutic levels. This is especially important for a therapeutic where sustained levels are necessary.

In another embodiment, a kit may comprise the therapeutic compositions described above. In a specific embodiment, the kit may comprise daily active dosage units comprising a therapeutic composition, herein the therapeutic composition allows for daily dosing regimen as prescribed, and wherein the initial administration of the therapeutic composition establishes its effect on bacterial vaginosis.

In another embodiment a kit may comprise the therapeutic composition of the present invention as well as an antibiotic formulation that is standard of care, thus having the ability to provide both antibiotic and therapeutic composition for daily dosage regimen.

In an embodiment methods are provided for the use of the therapeutic compositions for the treatment of bacterial vaginosis.

In an embodiment the invention treats BV with selective nutritional deprivation of vaginal nutrients that are essential only to bacteria associated with the BV condition, thus depressing their growth. Simultaneously, it selectively enhances the vaginal environment with nutrients that specifically promote the growth of protective lactobacilli modifying the vaginal environment to only support the protective bacterial growth and help restore the normal vaginal microbiome.

In an embodiment compositions are provided for treating a vaginal dysbiotic condition which is characterized by an elevated level of calprotectin detected in vaginal fluid.

In some embodiments, the therapeutic composition comprises manganese, either alone or in combination with zinc, as an active pharmaceutical ingredient The dose of manganese may be any amount of manganese, whether or not in combination with zinc or other active ingredients, sufficient to provide pharmacological effect. In some embodiments, the dosage of manganese is at least 2 micrograms, at least 5 micrograms, at least 10 micrograms, at least 20 micrograms, at least 30 micrograms or at least 50 micrograms. As will be appreciated, the dose level may be formulated to provide a sustained elevated level of manganese for some period of time in the vaginal environment and will also depend on how quickly the administration formulation releases the manganese, the ability of the bacteria to absorb the manganese and their native storage abilities to store manganese for periods of relative manganese shortage. Dosage level may also be dependent on the level of severity of dysbiotic condition to be treated, for example whether the dysbiotic condition includes a relatively low level of elevated calprotectin or relatively high level of elevated calprotectin in vaginal fluid (e.g., condition associated with moderate elevation around 3 μM calprotectin vs a condition with higher level of calprotectin at 100 M or more). The dosage of manganese may also depend on whether or not the composition also includes zinc. An upper limit of manganese dosage is dependent only on avoiding buildup of toxic levels in vaginal fluid. Manganese generally has low toxicity, and therefore very high dosages of manganese may be administered if desired, and with a relatively quick release profile to quickly counteract calprotectin binding and provide an assured excess of manganese to support growth of beneficial lactobacilli. However, because of a relatively quick turnover in the vaginal environment, a large excess of manganese might lead to significant waste. More practically, in some embodiments the dose of manganese may often be not more than 2000 micrograms, not more than 1000 micrograms, not more than 800 micrograms or even not more than 600 micrograms. In general, high doses of manganese are permissible even in the presence of high doses of zinc, although for economy dosage levels need not be excessive to that required for a desired therapeutic effect.

In some embodiments, the therapeutic composition comprises zinc as an active pharmaceutical ingredient, whether or not in combination with manganese.

The dose of zinc may be any amount of zinc, whether or not in combination with manganese and/or other active ingredients, sufficient to provide pharmacological effect. In some embodiments, the dosage of zinc is at least 4 micrograms, at least 10 micrograms, at least 25 micrograms, at least 50 micrograms of zinc or at least 100 micrograms. As will be appreciated, similar to the discussion concerning manganese, the dose level of zinc may be formulated to provide a sustained elevated level of zinc for some period of time in the vaginal environment and will also depend on how quickly the administration formulation releases the zinc. Dosage level may also be dependent on the level of severity of dysbiotic condition to be treated, for example whether the dysbiotic condition includes a relatively low level of elevated calprotectin or relatively high level of elevated calprotectin in vaginal fluid (e.g., condition associated with moderate elevation around 3 M calprotectin vs a condition with higher level of calprotectin at 100 μM or more). The dosage of manganese may also depend on whether or not the composition also includes zinc. An upper limit of zinc dosage is dependent only on avoiding buildup of toxic levels in vaginal fluid. Zinc generally has low toxicity, and therefore very high dosages of zinc may be administered if desired, and with a relatively quick release profile to quickly counteract calprotectin binding and provide an assured excess of zinc to support growth of beneficial lactobacilli. However, because of a relatively quick turnover in the vaginal environment, a large excess of zinc might lead to significant waste. More practically, in some embodiments the dose of zinc may often be not more than 5000 micrograms.

When a therapeutic composition includes both manganese and zinc, in some embodiments the dosage levels of each in the composition may be as summarized above for each individually, although if desired, the dosage of one or both of manganese and zinc may be reduced to take into account the dosage level of the other to provide sufficient dosages of each to adequately counteract calprotectin binding capacity.

### EXAMPLES

### Example 1: Concentration of Mn and Zn in Vaginal Fluid Compared to Metal Binding Sites in Calprotectin in Vaginal Fluid

Pairs of vaginal swabs were collected from 15 women and stored frozen. The swabs were centrifuged for 15 minutes using a Costar SpinX 0.22 uM filter to separate bacteria from vaginal fluid. The bacteria were washed from the filter using 500 μL of PBS/0.05% Proclin. The bacterial species in the vaginal fluids were identified and the bacterial vaginosis (BV) status of each sample was determined by PCR (AusDiagnostics Vaginitis and Vaginosis 12-well assay ref 87124), which measures L. crispatus, L jensenii, L gasseri, L iners, Gardnerella vaginalis and Atopbium vaginae. Bacterial counts were normalized to human epithelial cell marker in the same samples. The concentration of calprotectin in vaginal fluid was determined by ELISA (QUANTA Lite 704860. The concentration of both manganese (Mn) and zinc (Zn) in the vaginal fluid were determined by ICP-MS.

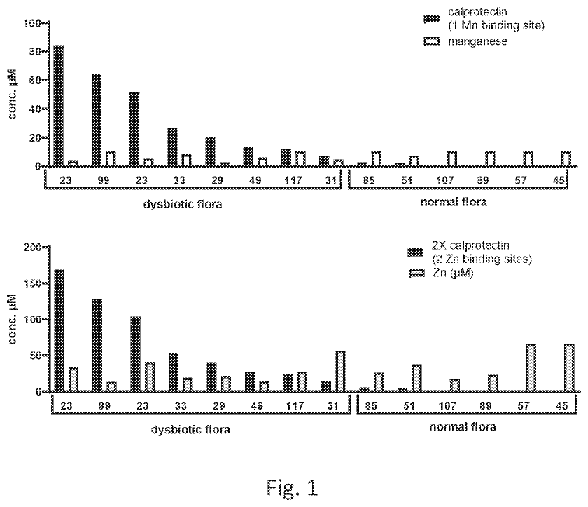

The data from the sample analysis is shown in Table 1 below. The levels of Mn and Zn metals found in vaginal fluid are not different in normal flora vs dysbiosis as shown in Table 1 and illustrated in FIG. 1. Elevated calprotectin levels result in not enough manganese for beneficial lactobacillus to grow/thrive. Calprotectin has 2 transition metal binding sites, one than can bind zinc and one that can bind zinc or manganese, and reportedly binds zinc with higher affinity. Thus, manganese or zinc to displace manganese from calprotectin, are possible treatment strategies.

Normal and dysbiotic flora are defined from the data in Table 1. As illustrated in FIG. 2 normal flora is characterized as being dominated by beneficial or good lactobacilli which includes L crispatus, L gasseri, and L jensenii in this assay. Dysbiotic flora is defined as Gardnerella vaginalis and/or the transitional species L iners, which unlike other lactobacilli, required iron instead of manganese for growth. In a healthy vaginal microbiome having normal flora, the concentration of Mn in vaginal fluid (measured by ICP-MS) is in excess over calprotectin binding sites (measured by ELISA). In a dysbiotic vaginal microbiome, Mn is limiting and the environment is not good for beneficial lactobacilli. Levels of zinc in vaginal fluid are not sufficient to overcome calprotectin and to allow free Mn in the dysbiotic samples. It was determined that sample #9 was an outlier and that sample was further determined to contain low levels of the yeast candida.

TABLE 1

| | Calprotectin in | | Metals in | | | | | | | | | |
|---|---|---|---|---|---|---|---|---|---|---|---|---|
| | vaginal fluid | | vaginal fluid | | | | | | | | | |
| | | 2X | | | Mn + | Relative copy numbers of various bacterial species on vaginal swab | | | | | | |
| Subject | CP | CP | Mn | Zn | Zn | L | L | L | total Good | L | Gardnerella | total bad |
| # | (μM) | (μM) | (μM) | (μM) | (μM) | crispatus | gasseri | jensenii | Lactobacilli | iners | vaginalis | bacteria |
| 9* | 101.7 | 203.4 | 8.8 | 51.3 | 60.1 | 0 | 0 | 0 | 0 | 0 | 0 | 0 |
| 23 | 84.46 | 168.92 | 4.2 | 33.2 | 37.4 | 1.58 | 0 | 0 | 1.58 | 1.75 | 1.05 | 2.79 |
| 99 | 64.11 | 128.22 | 10.2 | 13.2 | 23.4 | 0 | 7.75 | 0 | 7.75 | 0 | 21.33 | 21.33 |
| 27 | 51.83 | 103.67 | 5.1 | 40.8 | 45.9 | 0.75 | 0.03 | 0.24 | 1.02 | 4.29 | 483.76 | 488.05 |
| 33 | 26.31 | 52.62 | 8.4 | 19 | 27.4 | 0 | 0 | 0 | 0 | 4.43 | 1.17 | 5.61 |
| 29 | 20.27 | 40.53 | 2.7 | 21.2 | 23.9 | 0 | 0 | 0 | 0 | 0.75 | 41.93 | 42.68 |
| 49 | 13.56 | 27.11 | 6.2 | 13.5 | 19.7 | 0 | 0 | 0.16 | 0.16 | 2.23 | 15.07 | 17.3 |
| 117 | 11.78 | 23.56 | 10.2 | 26.5 | 36.7 | 0 | 0.01 | 0 | 0.01 | 9.06 | 0 | 9.06 |
| 31 | 7.32 | 14.64 | 4.5 | 56.2 | 60.7 | 0 | 0 | 0 | 0 | 38.72 | 107.07 | 145.79 |
| 85 | 2.74 | 5.48 | 10.1 | 25.8 | 35.9 | 0 | 5.52 | 0 | 5.52 | 13.29 | 0 | 13.29 |
| 51 | 2.31 | 4.62 | 7.2 | 37.3 | 44.5 | 221.74 | 0.43 | 0.01 | 222.18 | 0.01 | 0 | 0.01 |
| 107 | 0.82 | 1.64 | 10.2 | 16.5 | 26.7 | 68.32 | 0.1 | 0.04 | 68.46 | 0 | 0 | 0 |
| 89 | 0.55 | 1.1 | 10.1 | 22.8 | 32.9 | 72.67 | 0.16 | 2.09 | 74.92 | 0 | 0 | 0 |
| 57 | 0.12 | 0.25 | 10.1 | 65.4 | 75.5 | 52.17 | 0.07 | 0 | 52.24 | 0.02 | 0 | 0.02 |
| 45 | 0.00 | 0.00 | 10.1 | 65.4 | 75.5 | 100.62 | 0.12 | 0 | 100.74 | 0 | 0 | 0.00 |

*low levels of candida

### Example 2: Growth Curves for L. crispatus and L iners in Aerobic, Microaerophilic and Anerobic Conditions

L. crispatus (strain ATCC 33820) and L iners (strain ATCC 55195) were grown in NYCIII media+horse serum at 37° C. under the following conditions: aerobic (˜20% O2), microaerophilic (8-9% O2, 7-8% CO2) or anaerobic conditions (<0.1% O2, 8-16% CO2).

An OD 600 of each sample was measured at the following timepoints: 0, 8, 16, 24, 40, 48, and 52 hours.

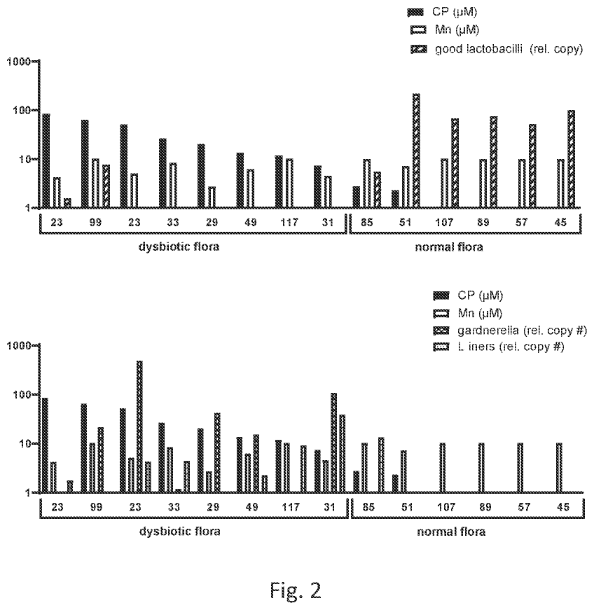

It is known that BV is a reducing environment having a low oxidation reduction potential (ORP), in contrast a healthy vaginal microbiome is an oxidizing environment, due in part to hydrogen peroxide produced from good lactobacilli. The vaginal microbiome is a microaerophilic environment but data shows L crispatus (good lactobacillus, uses Mn) can grow in aerobic conditions while L iners (transitional to BV, requires Fe) cannot as illustrated in FIG. 3. Some good lactobacilli strains use manganese superoxide dismutase (MnSOD) to thrive, others like L. crispatus can use just high levels of Mn, as L. crispatus does not have MnSOD. This observation can be exploited by making sure L. crispatus has enough Mn to thrive thus overcoming calprotectin in BV. Furthermore, it is hypothesized that including urea peroxide in formulations to increase hydrogen peroxide levels and oxidizing environment may favor lactobacilli strains that can utilize Mn or SOD.

### Example 3: Manganese Supplementation

The human vaginal microbiome is dominated by lactobacilli, which create an acidic environment thought to protect women against sexually transmitted pathogens and opportunistic infections. Bacterial vaginosis is a dysbiotic condition characterized by a shift from normal vaginal microbes predominantly lactobacilli in a low pH environment to anerobic species like Gardnerella vaginalis and Atopobium vaginae in an elevated pH environment. Our invention uses lactoferrin alone, manganese alone or lactoferrin and manganese in combination to create an environment where lactobacilli flourish and BV-associated bacteria cannot grow. Virtually all bacteria need iron, Lactobacilli are an exception and are not suppressed because they do not require iron, they are able to use manganese instead. This example demonstrates that manganese is limiting in BV thus providing the rationale for manganese supplementation as a treatment for BV.

Concentrations of 12-50 μg/mL elemental manganese are commonly used to culture lactobacilli species with 16 μg/mL (ATCC Medium: 416 Lactobacilli MRS Agar/Broth) used most commonly to grow strains such as Lactobacillus crispatus, a protective species in the vaginal microbiome. Inductively coupled mass spectrometry (ICP-MS) is used to measure elemental Mn in vaginal fluid from three women whose BV status was unknown. All had manganese levels significantly below that described in the literature as optimal for lactobacilli. Specifically, manganese levels were 0.047 μg/mL, <0.017 μg/mL and 0.039 μg/mL for donors T2407, T5659, and J4265.

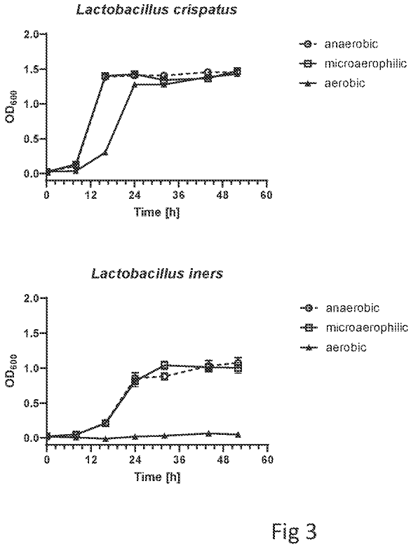

It was found that levels of manganese in vaginal fluid are low and are suppressed even further by the host immune response to BV-associated bacteria. Calprotectin, a protein highly expressed in neutrophils, contributes to this host defense by withholding Mn and Zn from invading pathogens. Levels of calprotectin in healthy women (Nugent Score 0-3) and women with BV (Nugent score 8-10) are compared. Vaginal swabs were collected, eluted with 1 mL of PBS, and residual solids were removed by centrifugation. Levels of calprotectin were measured by ELISA (QUANTA Lite® Calprotectin ELISA, Inova Diagnostics). As show in FIG. 4, calprotectin levels were 10-fold higher in women with BV than healthy women (median 223 μg/mL vs 22 μg/mL, p<0.001).

Taken together, the observation of low concentrations of manganese in vaginal fluid coupled with elevated calprotectin levels in bacterial vaginosis show a non-ideal environment for growth of lactobacillus and maintenance of a healthy vaginal microbiome and yield proof of concept for use of manganese for treatment of BV.

### Example 4: Manganese and Zinc Supplementation for BV and Maintenance of a Healthy Vaginal Microbiome

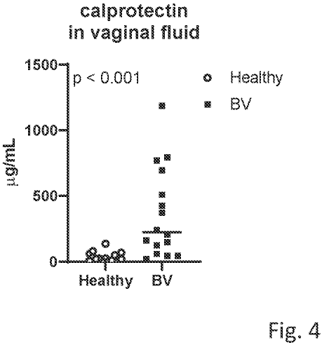

FIG. 5 confirms the observation that vaginal calprotectin levels are increased in women with BV and also demonstrates that calprotectin is elevated in women with L-iners dominated vaginal microbiomes relative to women with healthy microbiomes dominated by L crispatus. Sequential vaginal swabs were collected for PCR analysis of the vaginal microbiome (AusDiagnostics Vaginitis and Vaginosis 12-well test (REF87124) and 16S rRNA gene sequencing) and determination of calprotectin levels by ELISA (QUANTA Lite® Calprotectin ELISA, Inova Diagnostics). Vaginal fluid used for ELISA was isolated by centrifugation then diluted. Median concentrations of calprotectin in vaginal fluid were 57 μM in BV (n=24), 37 M in L. iners (n=28) and 10 μM in L. crispatus (n=31) with 95% confidence interval of calprotectin and L. iners dominated microbiomes overlapping with 95% confidence interval of calprotectin concentrations in BV. One molecule of calprotectin can bind 2 molecules of zinc or 1 molecule of manganese. When both Mn and Zn are present, Zn is preferentially bound. Levels of manganese and zinc were measured in vaginal fluid from women with unknown BV status. Vaginal swabs were centrifuged in Costar® Spin-X® Centrifuge Tube Filters, 0.22 μm Pore CA Membrane and the cell free fraction of vaginal fluid was analyzed by ICP-MS. The elevated levels of calprotectin in BV and L iners-dominated vaginal microbiomes were more than sufficient to bind all the available Mn and Zn in vaginal fluid. These results demonstrate that both BV and L iners create vaginal environments where L crispatus, which requires Mn not Fe for growth, cannot thrive. L iners is viewed as a transitional bacteria and women with L iners-dominated vaginal microbiomes are more likely to have a BV recurrence than women with L crispatus-dominated vaginal microbiomes. These results yield proof for the concept of manganese alone or in conjunction with zinc, for the treatment of BV. Furthermore, these results yield proof for the concept of manganese alone or in conjunction with zinc, for the prevention of recurrence of BV and maintenance of a healthy vaginal microbiome.

### Example 5: Doses of Manganese and Zinc for Treatment of BV and Maintenance of a Healthy Vaginal Microbiome

Lactobacillus crispatus has an absolute nutritional requirement for manganese. Elevated levels of calprotectin in BV and in L iners-dominated vaginal microbiomes sequester manganese and zinc, preventing growth of L crispatus its maintenance of a healthy vaginal microbiome. Dose ranges for Mn and Zn supplementation were determined by modeling the amount of Mn and Zn delivered from slow release formulations over time and superimposing that model on actual measured concentrations of endogenous levels of Mn and Zn as well as amounts of Mn and Zn that calprotectin will sequester (data from Example 5 and FIG. 5). The pharmacokinetic profile of formulation Z2, which is shown for lactoferrin in FIG. 1C, was used to build the model for Mn and Zn levels in vaginal fluid. Six women each received 3 doses of formulation Z2 separated by 48 hours. Vaginal fluid swabs were collected at t=0, 2, 4, 8, 12, 18, 24, 30, 36, and 42 hours post dose and vaginal fluid concentrations of lactoferrin and mannitol were determined. Average concentrations for the combined 18 dose administrations were obtained based on calculation of Area Under Curve for the locally estimated scatterplot smoothing (Loess) profile of lactoferrin and mannitol concentrations assuming 6 mL volume of distribution. The average concentration of lactoferrin was 33 mg/mL and the average concentrations of mannitol was 20 mg/mL. Average concentrations of both substances correspond to 0.1 fraction or 10% of the administered dose, 300 mg lactoferrin and 195 mg mannitol per Z2 tablet respectively. The 0.1 factor was used to estimate the expected average elemental Mn concentration based on the initial dose per tablet.

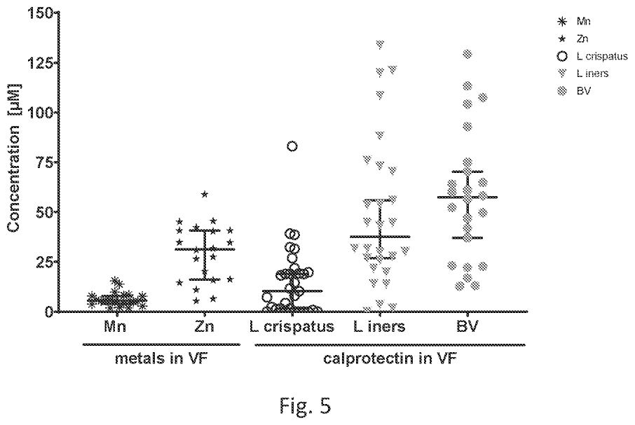

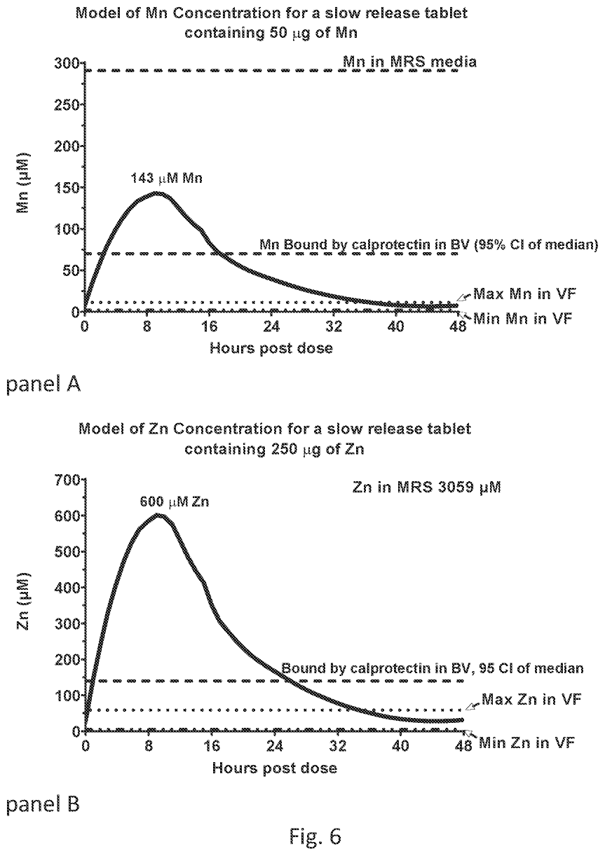

FIG. 6 panel A shows the results of modeling a 50 g dose of Mn included in a Z2 formulation. A 50 μg dose of Mn in yields a predicted maximal concentration (Cmax) of 143 μM Mn in vaginal fluid, which is ˜2 fold above the amount of Mn that could be bound by calprotectin in BV (95% CI of median is 70 M based on 1:1 Mn/CP) yet ˜2 fold below the concentration of Mn used in MRS media selective for lactobacillus. A 50 μg dose of Mn also fails to maintain for 24 hours levels Mn above that which can be sequestered by calprotectin (assuming no zinc available to displace Mn from calprotectin). These results indicate doses between the range of 25 and 500 μg of Mn in a slow release formulation are suitable for maintaining and healthy, L crispatus-dominated vaginal microbiome. It is also possible that even higher levels may provide additional benefit to the vaginal microbiota as other mechanisms of Mn utilization, sequestration or clearance have not been taken into account.

FIG. 6 panel B shows the results of modeling a 250 g dose of Zn included in a Z2 formulation. A 250 μg dose of Zn in yields a predicted maximal concentration (Cmax) of 600 μM Mn in vaginal fluid, which is ˜4 fold above the amount of Zn that could be bound by calprotectin in BV (95% CI of median is 140 M based on 2:1 Zn/CP) yet ˜5 fold below the concentration of Zn used in MRS media selective for lactobacillus. A 250 g dose of Zn also just minimally maintains Zn levels for 24 hours that which can be sequestered by calprotectin. These results indicate doses between the range of 250 and 5000 μg of Zn in a slow release formulation are suitable for maintaining and healthy, L crispatus-dominated vaginal microbiome. It is also possible that even higher levels of Zn may provide additional benefit to the vaginal microbiota as other mechanisms of Zn utilization, sequestration or clearance have not been taken into account.

### Example 6: Distribution of Total Calprotectin (CP) in Vaginal Fluid Samples

Vaginal samples were collected from 14 women using Copan 552C FLOQSwabs. Two swabs were collected from each woman, and after collection, the applicator shaft was cut and the swab tip was placed in a 0.22 μm Costar SpinX tube. The tube containing the sample placed in a centrifuge and spun at 16,000×G for 20 minutes at 4° C. The separated liquid fraction of the sample following centrifugation was isolated and the volume of the liquid was determined by weight.

To isolate the pellet fraction with separated solids following centrifugation, a filter cup containing the pellet was reversed and the pellet was transferred to a microcentrifuge tube by spinning the reversed filter in the microcentrifuge tube at 10,000×G for 5 min at 4° C. To wash extracellular calprotectin from the pellet, the pellet was resuspended in 300 μL of pe buffer (50 mM TRIS pH 7.4, 150 mM NaCl) by aspirating up and down using a pipetteman and mixed by vortexing. The microcentrifuge tube containing the resuspended pellet was spun at 10,000×G for 10 min at 4° C., and the pellet wash fraction was removed and reserved, leaving the pellet fraction. The pellet fraction was then extracted with 300 μL of 1× Calprotectin Extraction buffer provided in a Calprotectin ELISA kit to recover intracellular calprotectin remaining in the pellet fraction.

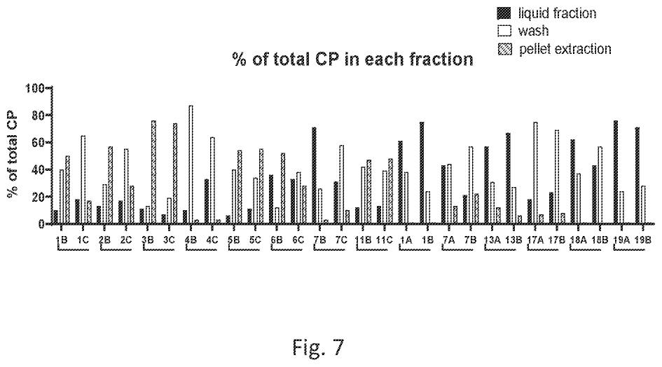

Concentrations of calprotectin in each fraction were determined by ELISA using a QUANTA Lite® Calprotectin Extended Range kit (704860) according to the manufacturer's instructions. The mass of calprotectin in each fraction was determined as the product of the concentrations as determined by ELISA and the volume of the fraction. The total calprotectin in each swab was determined by adding the mass of calprotectin in the liquid fraction, the pellet wash fraction, and the pellet extraction fraction. The percentage of total CP was determined by dividing the mass of CP in a given fraction by the total mass of CP. FIG. 7 shows the weight percent distribution of total assayed calprotectin between the different assayed fractions of the fluid sample.

As FIG. 7 illustrates, there is wide variation in which fractions the calprotectin is extracted from in samples collected from different women and even between 2 samples collected from the same woman. This precludes being able to accurately and reproducibly measure calprotectin levels in the specimen when an analysis is made from only part of a collected vaginal sample. It is important to measure both the extracellular and intracellular CP levels and relate them back to a measured size of the specimen or an identified part or parts of the specimen (e.g., pellet mass, neat separated liquid mass or volume or combination thereof) in order to provide maximum discrimination and enable clinical diagnosis.

### Example Implementation Combinations

Some other contemplated embodiments of implementation combinations for various aspects of this disclosure, with or without additional features as disclosed above or elsewhere herein, are summarized as follows:

### Compositions

1. A composition for use in the treatment of a vaginal dysbiotic condition with an elevated level of calprotectin in vaginal fluid, optionally as diagnosed with the method of any one of paragraphs 197-208, and alternatively or further optionally for use in a method of any one of paragraphs 78-175.2, the composition being formulated for intravaginal administration and the composition comprising:
a dose of essential metal for growth of vaginal flora Lactobacillus comprising at least one D-lactic acid-producing Lactobacillus, the essential metal being in a pharmaceutically acceptable form for intravaginal administration and the essential metal being selected from the group consisting of manganese, zinc and a combination of manganese and zinc;
optionally, such at least one D-lactic acid-producing Lactobacillus may include at least one member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof; preferably at least one member selected from the group consisting of Lactobacillus crispatus, Lactobacillus jensenii, Lactobacillus gasseri and combinations thereof; and more preferably Lactobacillus crispatus.

2. The composition of paragraph 1, wherein the dose of essential metal is in an amount to satisfy the following condition:

$$( ( 0.5 \times {Q}_{Zn} ) + {Q}_{Mn} ) \geq ( 0.001 \times K \times {C}_{ECp} )$$

wherein
Q_{Zn} is zero or a positive number equal to an amount of zinc in micromoles;
Q_{Mn} is zero or a positive number equal to an amount of manganese in micromoles;
C_{ECp} is a molar concentration of elevated calprotectin in vaginal fluid, in micromoles per liter;
K is a positive number of 1 or greater, and optionally an integer of 1 or greater; and
at least one of Q_{Zn} and Q_{Mn} is a positive number.

2.1 The composition of either one of paragraph 1 or paragraph 2, wherein C_{ECp} is at least 3, and optionally at least 5.

3. The composition of any one of paragraphs 1-2.1, wherein the dose of essential metal is in an amount to satisfy the following condition:

$$( ( 0.5 \times {Q}_{Zn} ) + {Q}_{Mn} ) \geq ( 0.1 \times K )$$

wherein
Q_{Zn} is the amount of zinc in micromoles;
Q_{Mn} is the amount of manganese in micromoles; and
K is a positive number of 1 or greater, and optionally an integer of 1 or greater.

4. The composition of either one of paragraph 2 or paragraph 3, wherein K is 1.

5. The composition of either one of paragraph 2 or paragraph 3, wherein K is 2.

6. The composition of either one of paragraph 2 or paragraph 3, wherein K is 3.

7. The composition of either one of paragraph 2 or paragraph 3, wherein K is 4.

8. The composition of either one of paragraph 2 or paragraph 3, wherein K is 5.

9. The composition of either one of paragraph 2 or paragraph 3, wherein K is 6.

10. The composition of either one of paragraph 2 or paragraph 3, wherein K is 7.

11. The composition of either one of paragraph 2 or paragraph 3, wherein K is 10 or larger.

12. The composition of any one of paragraphs 2-11, wherein K is not larger than 500.

### Manganese Levels in Compositions

13. The composition of any one of paragraphs 1-12, comprising manganese in an amount selected from the group consisting of at least 2 micrograms, at least 5 micrograms, at least 10 micrograms, at least 20 micrograms, at least 30 micrograms and at least 50 micrograms.

14. The composition of any one of paragraphs 1-13, comprising manganese up to an amount selected from the group consisting of 2000 micrograms and 1000 micrograms.

15. The composition of any one of paragraphs 1-14, comprising manganese in the form of a salt, optionally selected from the group consisting of a gluconate salt, a sulfate salt, a chloride salt, a citrate salt, a picolinate salt, a fatty acid salt and combinations thereof, with one preferred manganese salt being a sulfate salt.

16. The composition of any one of paragraphs 1-15, comprising manganese in the form of chelate, optionally an amino acid chelate, and optionally the chelate is selected from the group consisting of manganese bisglycinate chelate, manganese glycinate chelate, manganese aspartate and combinations thereof.

17. The composition of any one of paragraphs 1-16, comprising manganese in the form of manganese(II).

18. The composition of any one of paragraphs 1-12, wherein the composition comprises zinc and the composition is in the absence of manganese (e.g., Q_{Mn}=0).

### Zinc Level in Compositions

19. The composition of any one of paragraphs 1-18, comprising zinc in an amount of at least 4 micrograms, at least 10 micrograms, at least 25 micrograms, at least 50 micrograms of zinc or at least 100 micrograms.

20. The composition of any one of paragraphs 1-19, comprising zinc in an amount up to 5000 micrograms.

21. The composition of any one of paragraphs 1-20, comprising zinc in the form of a salt or a chelate, and preferably a salt and more preferably a sulfate salt.

22. The composition of any one of paragraphs 1-21, comprising zinc in the form of zinc(II).

23. The composition of any one of paragraphs 1-17, wherein the composition comprises manganese and is in the absence of zinc (e.g., Q_{Zn}=0).

### Probiotic in Compositions

24. The composition of any one of paragraphs 1-23, comprising a probiotic;
preferably, the probiotic comprises at least one D-lactic acid-producing Lactobacillus species, optionally; the at least one D-lactic acid-producing Lactobacillus species comprises a member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof,
more preferably the at least one D-lactic acid-producing Lactobacillus species comprises a member selected from the group consisting of Lactobacillus crispatus, Lactobacillus jensenii, Lactobacillus gasseri and combinations thereof; and
even more preferably the at least one D-lactic acid-producing Lactobacillus comprising Lactobacillus crispatus.

25. The composition of paragraph 24, comprising the probiotic in an amount of at least 10⁶, preferably at least 10⁷, more preferably at least 10⁸ and even more preferably at least 10⁹ colony forming units (CFU).

### Lactoferrin in Compositions

26. The composition of any one of paragraphs 1-25, comprising lactoferrin; optionally the lactoferrin is native lactoferrin, as a preferred option the lactoferrin is iron-depleted lactoferrin.

27. The composition of paragraph 26, comprising the lactoferrin in an amount of at least 10 milligrams, at least 25 milligrams, or at least 50 milligrams.

28. The composition of either one of paragraph 26 or paragraph 27, comprising the lactoferrin in an amount up to 500 milligrams.

29. The composition of any one of paragraphs 26-28, wherein the lactoferrin is bovine lactoferrin.

30. The composition of any one of paragraphs 26-29, wherein the lactoferrin is iron-depleted lactoferrin comprising a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

31. The composition of any one of paragraphs 26-30, wherein the lactoferrin is iron-depleted lactoferrin comprising a level of saturation with bound iron of at least 0.2 percent, and often at least 0.5 percent.

### Lipocalin-2 in Compositions

32. The composition of any one of paragraphs 1-31, comprising lipocalin-2.

33. The composition of paragraph 32, comprising the lipocalin-2 in an amount of at least 0.3 milligrams.

34. The composition of either one of paragraph 32 or paragraph 33, comprising the lipocalin-2 in an amount up to 30 milligrams.

### Oxidizing Agent in Compositions

35. The composition of any one of paragraphs 1-34, comprising an oxidizing agent, preferably hydrogen peroxide and more preferably hydrogen peroxide in the form of hydrogen peroxide-urea (urea peroxide).

36. The composition of paragraph 35, comprising the oxidizing agent in an amount formulated in the composition with a release profile to provide an increase in oxidation-reduction potential (ORP), relative to a standard hydrogen electrode, of vaginal fluid of at least 50 mV, at least 100 mV or even at least 150 mV.

37. The composition of either one of paragraph 35 or paragraph 36, comprising the oxidizing agent in an amount formulated in the composition with a release profile to provide at an increase in oxidation-reduction potential (ORP), relative to a standard hydrogen electrode, of vaginal fluid of not larger than 500 mV, not larger than 400 mV, not larger than 300 mV, not larger than 250 mV or even not larger than 200 mV.

### Acidification Agent in Compositions

38. The composition of any one of paragraphs 1-37, comprising an acidification agent to help lower pH of the vaginal environment, optionally the acidification agent is selected from the group consisting of boric acid, lactic acid and a lactate (e.g., calcium lactate or magnesium lactate), and more preferably the acidification agent is selected from the group consisting of lactic acid and a lactate, and even more preferably is a lactate.

38.1 The composition of paragraph 38, wherein the acidification agent is in an amount to lower pH of the vaginal environment to pH 4.5 or lower, and preferably not below pH 3.0.

39. The composition of either one of paragraph 38 or paragraph 38.1, comprising the acidification agent in an amount of at least 100 milligrams, optionally at least 200 milligrams.

40. The composition of either one of paragraph 38 paragraph 39, comprising the acidification agent in an amount up to 600 milligrams.

### Carbohydrate Source in Compositions

40.1 The composition of any one of paragraphs 1-40, comprising a carbohydrate source for the D-lactic acid producing Lactobacillus, optionally a sugar with the sugar optionally comprising a member selected from the group consisting of sucrose and lactose, and preferably the carbohydrate source comprises lactose.

40.2 The composition of paragraph 40.1, comprising the carbohydrate source in an amount formulated in the composition with a release profile to provide a concentration of the carbohydrate source in vaginal fluid of at least 0.25% wt/vol.

40.3 The composition of either one of paragraph 40.1 or 40.2, comprising the carbohydrate source in an amount formulated in the composition with a release profile to provide a concentration of the carbohydrate source in vaginal fluid of up to 3% wt/vol.

### Excipient in Compositions

41. The composition of any one of paragraphs 1-40.3, comprising at least one pharmaceutically acceptable excipient.

42. The composition of paragraph 41, wherein the at least one pharmaceutically acceptable excipient comprises at least one member selected from the group consisting of a filler, a gelling agent, a lubricant, glidant, a binder, a disintegrant and combinations thereof.

### Administration Forms of Compositions

43. The composition of any one of paragraphs 1-42, wherein the composition is in an intravaginally administrable form selected from the group consisting of a vaginal suppository tablet, vaginal ring or other vaginal depot, a vaginal capsule, vaginal cream and a vaginal gel.

43.1 The composition of any one of paragraphs 1-43, comprising a single said dose of essential metal.

43.2 The composition of any one of paragraphs 1-43.1, comprising a plurality of said doses of essential metal, and wherein the composition is divisible into a plurality of separate aliquots each comprising a said dose of essential metal (e.g., when the composition is in the form of a vaginal cream or vaginal gel contained in a tube or other receptacle from which the separate aliquots can be sequentially removed for separate administration).

43.3 The composition of any one of paragraphs 1-43.2, wherein the composition is formulated to release the essential metal of the dose after intravaginal administration for therapeutic effect over a period of time of at least 2 hours, optionally at least 4 hours, preferably at least 8 hours, more preferably at least 12 hours, even more preferably at least 24 hours.

### Kits

44. A kit for use in the treatment of a vaginal dysbiotic condition with an elevated level of vaginal calprotectin, the kit comprising;
a plurality of administration units, the plurality of administration units comprising at least one essential metal administration unit and preferably a plurality of essential metal administration units formulated for intravaginal administration, each said essential metal administration unit comprising a dose of essential metal for growth of at least one vaginal flora Lactobacillus comprising at least one D-lactic acid-producing Lactobacillus;
optionally, such D-lactic acid-producing Lactobacillus may include at least one member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof; preferably at least one member selected from the group consisting of Lactobacillus crispatus, Lactobacillus jensenii, Lactobacillus gasseri and combinations thereof; and more preferably Lactobacillus crispatus,
the essential metal being in a pharmaceutically acceptable form; and
the essential metal being selected from the group consisting of manganese, zinc and a combination of manganese and zinc;
and optionally at least one, preferably at least a plurality and more preferably all, of the essential metal administration units comprise a composition according to any one of paragraphs 1-43.3.

45. The kit of paragraph 44, comprising at least a number of said essential metal administration units selected from the group consisting of 2, 3, 4, 5, 7, 10 and 14.

46. The kit of either one of paragraph 44 or paragraph 45, comprising a number of said essential metal administration units not larger than a number selected from the group consisting of 100, 50, 30 and 15; and with one preferred range for the number of metal administration units being in a range of from 7 to 30.

### Probiotic in Kits

47. The kit of any one of paragraphs 44-46, comprising in at least one said administration unit, and preferably in a plurality of said administration units, a probiotic;
preferably, the probiotic comprises at least one D-lactic acid-producing Lactobacillus species, optionally the at least one D-lactic acid-producing Lactobacillus species comprises a member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof;
more preferably the at least one D-lactic acid-producing Lactobacillus species comprises a member selected from the group consisting of Lactobacillus crispatus, Lactobacillus jensenii, Lactobacillus gasseri and combinations thereof; and
even more preferably the at least one D-lactic acid-producing Lactobacillus comprising Lactobacillus crispatus.

48. The kit of paragraph 47, wherein at least one, and preferably a plurality, of said essential metal administration units comprise the probiotic.

49. The kit of either one of paragraph 47 or paragraph 48, comprising at least one probiotic administration unit, and preferably a plurality of the probiotic administration units, each said probiotic administration unit being a said administration unit different than a said essential metal administration unit, and each said probiotic administration unit comprising the probiotic, and optionally the kit comprises the same number or a different number of the probiotic administration units as a number of the essential metal administration units.

49.1 The kit of paragraph 49, wherein at least one said probiotic administration unit, and optionally each of at least a plurality said probiotic administration units or each and every one of the probiotic administration units is formulated for oral administration.

49.2 The kit of either one of paragraph 49 or paragraph 49.1, wherein at least one said probiotic administration unit, and optionally each of at least a plurality said probiotic administration units or each and every one of the probiotic administration units is formulated for intravaginal administration.

50. The kit of any one of paragraphs 47-49.2, wherein each said administration unit comprising the probiotic comprises the probiotic in an amount of at least 10⁶, preferably at least 10⁷, more preferably at least 10⁸ and even more preferably at least 10⁹ colony forming units (CFU).

### Lactoferrin in Kits

51. The kit of any one of paragraphs 44-50, comprising in at least one said administration unit, and preferably in a plurality of said administration units, lactoferrin, preferably bovine lactoferrin, optionally a native lactoferrin, and preferably an iron-depleted lactoferrin, and wherein each said lactoferrin unit is formulated for intravaginal administration.

52. The kit of paragraph 51, wherein at least one, and preferably a plurality, of said essential metal administration units comprise the lactoferrin.

53. The kit of either one of paragraph 51 or paragraph 52, comprising at least one lactoferrin administration unit, and preferably a plurality of the lactoferrin administration units, each said lactoferrin administration unit being a said administration unit different than a said essential metal administration unit, and each said lactoferrin administration unit comprising the lactoferrin, and optionally the kit comprises the same number or a different number of the lactoferrin administration units as a number of the essential metal administration units.

54. The kit of any one of paragraphs 51-53, wherein each said administration unit comprising lactoferrin comprises the lactoferrin in an amount of at least 10 milligrams, at least 25 milligrams, or at least 50 milligrams.

55. The kit of any one of paragraphs 51-54, wherein each said administration unit comprising lactoferrin comprises the iron-depleted lactoferrin in an amount up to 500 milligrams.

56. The kit of any one of paragraphs 51-55, wherein the lactoferrin is bovine lactoferrin.

57. The kit of any one of paragraphs 51-56, wherein the lactoferrin is iron-depleted lactoferrin comprising a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

58. The kit of any one of paragraphs 51-57, wherein the lactoferrin is iron-depleted lactoferrin comprising a level of saturation with bound iron of at least 0.2 percent, and often at least 0.5 percent.

### Lipocalin-2 in Kits

59. The kit of any one of paragraphs 44-58, comprising in at least one said administration unit, and preferably in a plurality of said administration units, lipocalin-2, and wherein each said administration unit comprising lipocalin-2 is formulated for intravaginal delivery.

60. The kit of paragraph 59, wherein at least one, and preferably a plurality, of said essential metal administration units comprise lipocalin-2.

61. The kit of either one of paragraph 59 or paragraph 60, comprising at least one lipocalin-2 administration unit, and preferably a plurality of the lipocalin-2 administration units, each said lipocalin-2 administration unit being a said administration unit different than a said essential metal administration unit, and each said lipocalin-2 administration unit comprising lipocalin-2, and optionally the kit comprises the same number or a different number of the lipocalin-2 administration units as a number of the essential metal administration units.

62. The kit of any one of paragraphs 59-61, wherein each said administration unit comprising lipocalin-2 comprises lipocalin-2 in an amount of at least 0.3 milligrams.

63. The kit of any one of paragraphs 59-62, wherein each said administration unit comprising lipocalin-2 comprises lipocalin-2 in an amount up to 30 milligrams.

### Oxidizing Agent in Kits

64. The kit of any one of paragraphs 44-63, comprising in at least one said administration unit, and preferably in a plurality of said administration units, an oxidizing agent, preferably hydrogen peroxide and more preferably hydrogen peroxide in the form of hydrogen peroxide-urea (urea peroxide), and wherein each said administration unit comprising the oxidizing agent is formulated for intravaginal delivery.

65. The kit of paragraph 64, wherein at least one, and preferably a plurality, of said essential metal administration units comprise the oxidizing agent.

66. The kit of either one of paragraph 64 or paragraph 65, comprising at least one oxidizing agent administration unit, and preferably a plurality of the oxidizing agent administration units, each said oxidizing administration unit being a said administration unit different than a said essential metal administration unit, and each said oxidizing agent administration unit comprising the oxidizing agent, and optionally the kit comprises the same number or a different number of the oxidizing agent administration units as a number of the essential metal administration units.

67. The kit of any one of paragraphs 64-66, wherein each said administration unit comprising the oxidizing agent comprises the oxidizing agent in an amount formulated in the administration unit with a release profile to provide an increase in oxidation-reduction potential (ORP), relative to a standard hydrogen electrode, of vaginal fluid of at least 50 mV, at least 100 mV or even at least 150 mV.

68. The kit of any one of paragraphs 64-67, wherein each said administration unit comprising the oxidizing agent comprises the oxidizing agent in an amount formulated in the administration unit with a release profile to provide at an increase in oxidation-reduction potential (ORP), relative to a standard hydrogen electrode, of vaginal fluid by not larger than 500 mV, not larger than 400 mV, not larger than 300 mV, not larger than 250 mV or even not larger than 200 mV.

### Acidification Agent in Kits

69. The kit of any one of paragraphs 44-68, comprising in at least one said administration unit, and preferably in a plurality of said administration units, an acidification agent to help lower pH of the vaginal environment, and preferably to lower the pH of the vaginal environment to a pH of 4.5 or lower and preferably not below pH 3.0, optionally the acidification agent is selected from the group consisting of boric acid, lactic acid and a lactate (e.g., calcium lactate or magnesium lactate), preferably the acidification agent is selected from the group consisting of lactic acid and a lactate, and more preferably the acidification agent is a lactate, and wherein each said administration unit comprising the acidification agent is formulated for intravaginal delivery.

70. The kit of paragraph 69, wherein at least one, and preferably a plurality, of said essential metal administration units comprise the acidification agent.

71. The kit of either one of paragraph 69 or paragraph 70, comprising at least one acidification agent administration unit, and preferably a plurality of the acidification agent administration units, each said acidification agent administration unit comprising the acidification agent, and optionally the kit comprises the same number or a different number of the acidification agent administration units as a number of the essential metal administration units.

72. The kit of any one of paragraphs 69-71, wherein each said administration unit comprising the acidification agent comprises the acidification agent in an amount of at least 100 milligrams and optionally at least 200 milligrams.

73. The kit of any one of paragraphs 69-72, wherein each said administration unit comprising the acidification agent comprises the acidification agent in an amount up to 600 milligrams, or up to 500 milligrams.

### Carbohydrate Source in Kits

73.1. The kit of any one of paragraphs 44-73, comprising in at least one said administration unit, and preferably in a plurality of said administration units, a carbohydrate source for the D-lactic acid producing Lactobacillus, optionally a sugar with the sugar optionally comprising a member selected from the group consisting of sucrose and lactose, and preferably the carbohydrate source comprises lactose, and wherein each said administration unit comprising the carbohydrate source is formulated for intravaginal delivery.

73.2. The kit of paragraph 73.1, wherein at least one, and preferably a plurality, of said essential metal administration units comprise the carbohydrate source.

73.3. The kit of either one of paragraph 73.1 or paragraph 73.2, comprising at least one carbohydrate source administration unit, and preferably a plurality of the carbohydrate source administration units, each said carbohydrate source administration unit comprising the carbohydrate source, and optionally the kit comprises the same number or a different number of the carbohydrate source administration units as a number of the essential metal administration units.

73.4. The kit of any one of paragraphs 73.1-73.3, wherein each said administration unit comprising the carbohydrate source comprises carbohydrate source in an amount formulated in the composition with a release profile to provide a concentration of the carbohydrate source in vaginal fluid of at least 0.25% wt/vol.

73.5. The kit of any one of paragraphs 73.1-73.4, wherein each said administration unit comprising the carbohydrate source comprises the carbohydrate source in an amount formulated in the composition with a release profile to provide a concentration of the carbohydrate source in vaginal fluid up to 3% wt/vol.

### Excipient in Kits

74. The kit of any one of paragraphs 44-73.5, comprising at least one pharmaceutically acceptable excipient.

75. The kit of paragraph 74, wherein the at least one pharmaceutically acceptable excipient comprises at least one member selected from the group consisting of a filler, a gelling agent, a lubricant, glidant, a binder, a disintegrant and combinations thereof.

### Forms of Kits

76. The kit of any one of paragraphs 44-75, wherein each said administration unit is in an intravaginally administrable form each independently selected from the group consisting of a vaginal suppository tablet, vaginal ring or other vaginal depot, a vaginal capsule, vaginal cream and a vaginal gel.

76.1 The kit of any one of paragraphs 44-76, comprising a vaginal applicator configured for intravaginal delivery of a said essential metal administration unit; and
optionally, the vaginal applicator comprises:
a barrel wherein the barrel has a distal end configured to be inserted into a vagina during intravaginal administration and a proximal end to be disposed outside of the vagina during intravaginal administration, the barrel has a lumen therethrough;
a receptacle volume adjacent a distal end of the barrel, and optionally including an interior space within the lumen, to receive a said essential metal administration unit; and
a plunger inserted into lumen through the proximal end of the barrel and configured to be translatable through the barrel to push the administration unit out of the receptacle and into the vagina during intravaginal administration.

77. The kit of any one of paragraphs 44-76.1, comprising a common packaging enclosure containing the plurality of the administration units, and optionally each said administration unit is individually contained in a separate enclosure, preferably a separate hermetically sealed enclosure, for example in a separate sealed cavity of a tray or blister pack comprising the plurality of the administration units.

### Methods of Treatment

78. A method of treating a person for a vaginal dysbiotic condition with an analytically-determined, elevated calprotectin concentration (C_{ECp}) in vaginal fluid, optionally as diagnosed with the method of any one of paragraphs 197-208, the method comprising:
intravaginal administration to the person of essential metal selected from the group consisting of zinc, manganese and combinations of zinc and manganese sufficient to achieve an essential metal loading ratio in vaginal fluid of 1.0 or larger, wherein the essential metal loading ratio is calculated by the equation

$${R}_{EM} = ( ( 0.5 \times {C}_{Zn} ) + {C}_{Mn} ) / {C}_{ECp}$$

wherein,
R_{EM} is the essential metal loading ratio;
C_{Zn} is zero or a positive number equal to a concentration in micromoles per liter of zinc in the vaginal fluid; and
C_{Mn} is zero or a positive number equal to a concentration in micromoles per liter of manganese in the vaginal fluid;
C_{ECp} is the analytically-determined elevated calprotectin concentration in micromoles per liter; and
at least one of C_{Zn} and C_{Mn} is a positive number.

79. The method of paragraph 78, wherein the essential metal loading ratio is 1.1 or larger, 1.2 or larger, 1.5 or larger, and preferably at least 2.0 or larger.

80. The method of either one of paragraph 78 or paragraph 79, comprising at least one metal administration event and preferably a plurality of temporally spaced metal administration events, each said metal administration event comprising intravaginal administration to the person of a dose of the essential metal.

81. The method of paragraph 80, wherein each said dose of the essential metal is sufficient to achieve for at least a period of time after the corresponding metal administration event the essential metal loading ratio in the vaginal fluid.

82. The method of paragraph 81, wherein the period of time is at least 2 hours, optionally at least 4 hours, preferably at least 8 hours, more preferably at least 12 hours, even more preferably at least 24 hours.

83. A method of treating a person for a vaginal bacteria dysbiotic condition with an elevated level of calprotectin in vaginal fluid, optionally as diagnosed with the method of any one of paragraphs 197-208, the method comprising:
intravaginal administration to the person an effective amount of essential metal to elevate manganese availability in the vagina to a level promoting growth in the presence of the calprotectin of at least one D-lactic acid-producing Lactobacillus species for a healthy vaginal microbiota;
wherein the essential metal is selected from the group consisting of manganese, zinc and combinations of manganese and zinc;
   preferably the at least one D-lactic acid-producing Lactobacillus species comprising a member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof;
more preferably the at least one D-lactic acid-producing Lactobacillus species comprising a member selected from the group consisting of Lactobacillus crispatus, Lactobacillus jensenii, Lactobacillus gasseri and combinations thereof; and
even more preferably the at least one D-lactic acid-producing Lactobacillus comprising Lactobacillus crispatus.

84. A method of treating a person for a vaginal dysbiotic condition with manganese starvation stress in the vagina from an elevated level of calprotectin in vaginal fluid inhibiting growth of at least one D-lactic acid-producing Lactobacillus species, optionally as diagnosed with the method of any one of paragraphs 197-208, the method comprising:
intravaginal administration to the person an effective amount of essential metal to partially or fully counteract the manganese starvation stress from the elevated calprotectin;
wherein the essential metal is selected from the group consisting of manganese, zinc and combinations of manganese and zinc; and
preferably the at least one D-lactic acid-producing Lactobacillus species comprising a member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof,
more preferably the at least one D-lactic acid-producing Lactobacillus species comprising a member selected from the group consisting of Lactobacillus crispatus, Lactobacillus jensenii, Lactobacillus gasseri and combinations thereof; and
even more preferably the at least one D-lactic acid-producing Lactobacillus comprising Lactobacillus crispatus.

85. A method of treating a person for a vaginal dysbiotic condition with elevated level of calprotectin in vaginal fluid, optionally as diagnosed with the method of any one of paragraphs 197-208, the method comprising:
intravaginal administration to the person an effective amount of essential metal for treatment of the vaginal dysbiotic condition;
wherein the essential metal is selected from the group consisting of manganese, zinc and combinations of manganese and zinc.

### Treatment Regimens—Essential Metal

86. The method of any one of paragraphs 78-85, comprising a treatment regimen including at least one metal administration event and preferably a plurality of temporally spaced metal administration events, each said metal administration event comprising intravaginal administration to the person of an effective dose of the essential metal;
preferably each said metal administration event comprises intravaginally administering a composition of any one of paragraphs 1-43.3, and
optionally, the person has an elevated calprotectin level in vaginal fluid at commencement of the treatment regimen, preferably as determined by an assay of vaginal fluid, selected from the group consisting of at least 3 micromoles per liter, at least 5 micromoles per liter, at least 10 micromoles per liter and at least 20 micromoles per liter.

87. The method of paragraph 86, comprising a plurality of the metal administration events and wherein the metal administration events are at a frequency of not more than twice per day, preferably not more than once per day;
optionally, the metal administration events are at a frequency of not less than once per week and preferably not more than once every 3 days; and
one preferred frequency for the metal administration events is once per day.

88. The method of either one of paragraph 86 or paragraph 87, comprising continuing the treatment regimen at least until a level of calprotectin in vaginal fluid has dropped below a threshold level, and optionally discontinuing the treatment regimen after the level of calprotectin in the vaginal fluid had dropped below the threshold level.

89. The method of paragraph 88, wherein the threshold level of calprotectin in the vaginal fluid is no larger than 3 micromoles per liter, preferably no larger than 2 micromoles per liter, even more preferably no larger than 1 micromole per liter and still more preferably no larger than 0.5 micromoles per liter.

89.1 The method of any one of paragraphs 86-89, comprising continuing the treatment regimen at least until, and optionally discontinuing the treatment regimen after, an assay of the vaginal fluid indicates vaginal microbiota dominated by D-lactic acid producing Lactobacillus.

89.2. The method of any one of paragraphs 86-89.1, comprising continuing the treatment regimen at least until, and optionally discontinuing the treatment after, a concentration of D-lactate in vaginal fluid is indicative of vaginal microbiota dominated by D-lactic acid-producing Lactobacillus.

89.3 The method of any one of paragraphs 86-89.2, comprising continuing the treatment regimen at least until, and optionally discontinuing the treatment regimen after, a concentration ratio of D-lactate to L-Lactate in vaginal fluid is indicative of vaginal microbiota dominated by D-lactic acid-producing Lactobacillus.

89.4 The method of any one of paragraphs 86-89.3, comprising continuing the treatment regimen at least until, and optionally discontinuing the treatment regimen after, a concentration acetate in vaginal fluid is indicative of vaginal microbiota not being dominated by bacteria associated with bacterial vaginosis (e.g., Gardnerella vaginalis, Atopobium vaginae, Prevotella species, Mobiluncus species).

90. The method of any one of paragraphs 86-89.4, comprising continuing the treatment regimen at least until a microbiota assay of vaginal fluid indicates elimination of the dysbiotic condition.

91. The method of any one of paragraphs 86-90, wherein each said metal administration event comprises intravaginal administration of a said essential metal administration unit of a kit according to any one of paragraphs 44-77

92. The method of any one of paragraphs 86-91, wherein each said metal administration event comprises intravaginal administration of an administration unit comprising the composition any one of paragraphs 1-43.3.

93. The method of any one of paragraphs 86-91, comprising at least a minimum number of the metal administration events selected from the group consisting of 2, 3, 4, 5, 7, 10 and 14, and optionally comprising a maximum number of the metal administration events selected from the group consisting of 100, 50, 30 and 15; and with one preferred range for the number of the metal administration events being in a range of from 7 to 30.

### Treatment Regimens—Probiotic

94. The method of any one of paragraphs 86-93, wherein the treatment regimen comprises at least one probiotic administration event, and preferably a plurality of probiotic administration events, each said probiotic administration event comprising administration of a probiotic;
preferably the probiotic comprises at least one D-lactic acid-producing Lactobacillus species;
optionally the at least one D-lactic acid-producing Lactobacillus species comprises a member selected from the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof,
more preferably the at least one D-lactic acid-producing Lactobacillus species comprises a member selected from the group consisting of Lactobacillus crispatus, Lactobacillus jensenii, Lactobacillus gasseri and combinations thereof; and
even more preferably the at least one D-lactic acid-producing Lactobacillus comprises Lactobacillus crispatus.

95. The method of paragraph 94, wherein at least one said probiotic administration event, and optionally each of a plurality of said probiotic administration events, comprises oral administration of the probiotic.

96. The method of either one of paragraph 94 or paragraphs 95, wherein at least one said probiotic administration event, and optionally each said probiotic administration event, comprises intravaginal administration of the probiotic.

97. The method of any one of paragraphs 94-96, wherein at least one said probiotic administration event, and optionally each of a plurality of said probiotic administration events, is contemporaneous with a said metal administration event.

98. The method of any one of paragraphs 94-97, wherein at least one said probiotic administration event, and optionally each of a plurality of said probiotic administration events, occurs after a said metal administration event, and further optionally on a different day than occurrence of a said metal administration event.

99. The method of any one of paragraphs 94-98, wherein each said probiotic administration event comprises administering a dose of probiotic in an amount of at least 10⁶, preferably at least 10⁷, more preferably at least 10⁸ and even more preferably at least 10⁹ colony forming units (CFU).

100. The method of any one of paragraphs 94-99, wherein the treatment regimen comprises at least a minimum number of the probiotic administration events selected from the group consisting of 2, 3, 4, 5, 7, 10 and 14, and optionally comprising a maximum number of the probiotic administration events selected from the group consisting of 100, 50, 30 and 15; and with one preferred range for the number of the probiotic administration events being in a range of from 7 to 30.

101. The method of paragraph 100, comprising a plurality of the probiotic administration events and wherein the probiotic administration events are at a frequency of not more than twice per day, preferably not more than once per day;
optionally, the probiotic administration events are at a frequency of not less than once per week and preferably not more than once every 3 days.

102. The method of either one of paragraph 100 or paragraph 101, wherein at least one said probiotic administration event, and preferably at least each of the minimum number of the probiotic administration events, occurs during a time commencing no earlier than one day prior to a first said metal administration event of the treatment regimen and ending not more than one day after a last said metal administration event of the treatment regimen.

103. The method of any one of paragraphs 94-102, wherein at least one said probiotic administration event, and preferably each of a plurality of said probiotic administration events, comprises intravaginal administration of the probiotic contained in a composition according to any one of paragraphs 1-43.3.

104. The method of any one of paragraphs 94-103, wherein at least one said probiotic administration event, and preferably each of a plurality of said probiotic administration events, comprises intravaginal administration of the probiotic contained in a said essential metal administration unit of the kit of any one of paragraphs 44-77.

105. The method of any one of paragraphs 94-104, wherein at least one said probiotic administration event, and preferably each of a plurality of said probiotic administration events, comprises administration of the probiotic contained in a said probiotic administration unit of the kit of any one of paragraphs 49-50.

### Treatment Regimens—Lactoferrin

106. The method of any one of paragraphs 86-105, wherein the treatment regimen comprises at least one lactoferrin administration event, and preferably a plurality of lactoferrin administration events, each said lactoferrin administration event comprising intravaginal administration of lactoferrin, and preferably an iron-depleted lactoferrin; and optionally at least one or each of a plurality of said oxidizing agent administration events is contemporaneous with a said probiotic administration event of any one of paragraphs 94-105.

107. The method of paragraph 106, wherein at least one said lactoferrin administration event, and optionally each of a plurality of said lactoferrin administration events, is contemporaneous with a said metal administration event.

108. The method of either one of paragraph 106 or paragraph 107, wherein at least one said lactoferrin administration event, and optionally each of a plurality of said lactoferrin administration events, occurs after a said metal administration event, and further optionally on a different day than occurrence of a said metal administration event.

109. The method of any one of paragraphs 106-108, wherein each said lactoferrin administration event comprises intravaginally administering a dose of lactoferrin in an amount of at least 10 milligrams, at least 25 milligrams or at least 50 milligrams.

110. The method of any one of paragraphs 106-109, wherein each said lactoferrin administration event comprises intravaginally administering a dose of lactoferrin in amount of up to 500 milligrams.

111. The method of any one of paragraphs 106-110, wherein the treatment regimen comprises at least a minimum number of the lactoferrin administration events selected from the group consisting of 2, 3, 4, 5, 7, 10 and 14, and optionally comprising a maximum number of the lactoferrin administration events selected from the group consisting of 100, 50, 30 and 15; and with one preferred range for the number of the lactoferrin administration events being in a range of from 7 to 30.

112. The method of paragraph 111, comprising a plurality of the lactoferrin administration events and wherein the lactoferrin administration events are at a frequency of not more than twice per day, preferably not more than once per day;
optionally, the lactoferrin administration events are at a frequency of not less than once per week and preferably not more than once every 3 days; and
one preferred frequency for the lactoferrin administration events is once per day; and
preferably the lactoferrin is at an elevated in vaginal fluid for the entire time duration between successive lactoferrin administration events.

113. The method of either one of paragraph 111 or paragraph 112, wherein at least one said lactoferrin administration event, and preferably at least each of the minimum number of the lactoferrin administration events, occurs during a time commencing no earlier than one day prior to a first said metal administration event of the treatment regimen and ending not more than one day after a last said metal administration event of the treatment regimen.

114. The method of any one of paragraphs 106-113, wherein at least one said lactoferrin administration event, and preferably each of a plurality of said lactoferrin administration events, comprises intravaginal administration of the lactoferrin contained in a composition according to any one of paragraphs 1-43.3.

115. The method of any one of paragraphs 106-114, wherein at least one said lactoferrin administration event, and preferably each of a plurality of said lactoferrin administration events, comprises intravaginal administration of the lactoferrin contained in a said essential metal administration unit of the kit of any one of paragraphs 44-77.

116. The method of any one of paragraphs 106-115, wherein at least one said lactoferrin administration event, and preferably each of a plurality of said lactoferrin administration events, comprises administration of the lactoferrin contained in a said lactoferrin administration unit of the kit of any one of paragraphs 53-58.

117. The method of any one of paragraphs 106-116, wherein the lactoferrin is iron-depleted lactoferrin comprising a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

118. The method of any one of paragraphs 106-117, wherein the lactoferrin is iron-depleted lactoferrin comprising a level of saturation with bound iron of at least 0.2 percent, and often at least 0.5 percent.

119. The method of any one of paragraphs 106-118, wherein the lactoferrin is bovine lactoferrin.

### Treatment Regimens—Lipocalin-2

120. The method of any one of paragraphs 86-119, wherein the treatment regimen comprises at least one lipocalin-2 administration event, and preferably a plurality of lipocalin-2 administration events, each said lipocalin-2 administration event comprising intravaginal administration of lipocalin-2, and optionally at least one or each of a plurality of said oxidizing agent administration events is contemporaneous with an administration event selected from the group consisting of a said probiotic administration event of any one of paragraphs 94-105, a said lactoferrin administration event of any one of paragraphs 106-119 administration event and combinations thereof.

121. The method of paragraph 120, wherein at least one said lipocalin-2 administration event, and optionally each of a plurality of said lipocalin-2 administration events, is contemporaneous with a said metal administration event.

122. The method of either one of paragraph 120 or paragraph 121, wherein at least one said lipocalin-2 administration event, and optionally each of a plurality of said lipocalin-2 administration events, occurs after a said metal administration event, and further optionally on a different day than occurrence of a said metal administration event.

123. The method of any one of paragraphs 120-122, wherein each said lipocalin-2 administration event comprises intravaginally administering a dose of lipocalin-2 in an amount of at least 10 milligrams, at least 25 milligrams or at least 50 milligrams.

124. The method of any one of paragraphs 120-123, wherein each said lipocalin-2 administration event comprises intravaginally administering a dose of lipocalin-2 in amount of up to 500 milligrams.

125. The method of any one of paragraphs 120-124, wherein the treatment regimen comprises at least a minimum number of the lipocalin-2 administration events selected from the group consisting of 2, 3, 4, 5, 7, 10 and 14, and optionally comprising a maximum number of the lipocalin-2 administration events selected from the group consisting of 100, 50, 30 and 15; and with one preferred range for the number of the lipocalin-2 administration events being in a range of from 7 to 30.

126. The method of paragraph 125, comprising a plurality of the lipocalin-2 administration events and wherein the lipocalin-2 administration events are at a frequency of not more than twice per day, preferably not more than once per day;
optionally, the lipocalin-2 administration events are at a frequency of not less than once per week and preferably not more than once every 3 days; and
one preferred frequency for the lipocalin-2 administration events is once per day.

127. The method of either one of paragraph 125 or paragraph 126, wherein at least one said lipocalin-2 administration event, and preferably at least each of the minimum number of the lipocalin-2 administration events, occurs during a time commencing no earlier than one day prior to a first said metal administration event of the treatment regimen and ending not more than one day after a last said metal administration event of the treatment regimen.

128. The method of any one of paragraphs 120-127, wherein at least one said lipocalin-2 administration event, and preferably each of a plurality of said lipocalin-2 administration events, comprises intravaginal administration of the lipocalin-2 contained in a composition according to any one of paragraphs 1-43.3.

129. The method of any one of paragraphs 120-128, wherein at least one said lipocalin-2 administration event, and preferably each of a plurality of said lipocalin-2 administration events, comprises intravaginal administration of the lipocalin-2 contained in a said essential metal administration unit of the kit of any one of paragraphs 44-77.

130. The method of any one of paragraphs 120-129, wherein at least one said lipocalin-2 administration event, and preferably each of a plurality of said lipocalin-2 administration events, comprises administration of the lipocalin-2 contained in a said lipocalin-2 administration unit of the kit of any one of paragraphs 61-63.

### Treatment Regimens—Oxidizing Agent

131. The method of any one of paragraphs 86-130, wherein the treatment regimen comprises at least one oxidizing agent administration event, and preferably a plurality of oxidizing agent administration events, each said oxidizing agent administration event comprising intravaginal administration of oxidizing agent, and optionally at least one or each of a plurality of said oxidizing agent administration events is contemporaneous with an administration event selected from the group consisting of a said probiotic administration event of any one of paragraphs 94-105, a said lactoferrin administration event of any one of paragraphs 106-119, a said lipocalin-2 administration event of any one of paragraphs 120-130 and combinations thereof.

132. The method of paragraph 131, wherein at least one said oxidizing agent administration event, and optionally each of a plurality of said oxidizing agent administration events, is contemporaneous with a said metal administration event.

133. The method of either one of paragraph 131 or paragraph 132, wherein at least one said oxidizing agent administration event, and optionally each of a plurality of said oxidizing agent administration events, occurs after a said metal administration event, and further optionally on a different day than occurrence of a said metal administration event.

134. The method of any one of paragraphs 131-133, wherein each said oxidizing agent administration event comprises intravaginally administering a dose of oxidizing agent and as a consequence increasing oxidation-reduction potential (ORP), relative to a standard hydrogen electrode, of vaginal fluid by at least 50 mV, at least 100 mV or even at least 150 mV;
preferably, not increasing the ORP of vaginal fluid to above 550 mV, not larger than 450 mV, not larger than 350 mV and more preferably not raising the ORP of vaginal fluid to above 300 mV; and
preferably, increasing the ORP of vaginal fluid from an initial ORP, prior to the intravaginally administering the dose of oxidizing agent, of not larger than 150 mV or not larger than 100 mV.

135. The method of any one of paragraphs 131-134, wherein each said oxidizing agent administration event comprises intravaginally administering a dose of oxidizing agent and as a consequence increasing oxidation-reduction potential (ORP), relative to a standard hydrogen electrode, of vaginal fluid by not more than 500 mV, not larger than 400 mV, not larger than 300 mV, not more than 250 mV or even not more than 200 mV.

136. The method of any one of paragraphs 131-135, wherein the treatment regimen comprises at least a minimum number of the oxidizing agent administration events selected from the group consisting of 2, 3, 4, 5, 7, 10 and 14, and optionally comprising a maximum number of the oxidizing agent administration events selected from the group consisting of 100, 50, 30 and 15; and with one preferred range for the number of the oxidizing agent administration events being in a range of from 7 to 30.

137. The method of paragraph 136, comprising a plurality of the oxidizing agent administration events and wherein the oxidizing agent administration events are at a frequency of not more than twice per day, preferably not more than once per day; and
optionally, the oxidizing agent administration events are at a frequency of not less than once per week and preferably not more than once every 3 days; and

138. The method of paragraph 137, wherein the frequency for the metal administration events is once per day.

139. The method of any one of paragraphs 136-138, wherein at least one said oxidizing agent administration event, and preferably at least each of the minimum number of the oxidizing agent administration events, occurs during a time commencing no earlier than one day prior to a first said metal administration event of the treatment regimen and ending not more than one day after a last said metal administration event of the treatment regimen.

140. The method of any one of paragraphs 131-139, wherein at least one said oxidizing agent administration event, and preferably each of a plurality of said oxidizing agent administration events, comprises intravaginal administration of the oxidizing agent contained in a composition according to any one of paragraphs 1-43.3.

141. The method of any one of paragraphs 131-140, wherein at least one said oxidizing agent administration event, and preferably each of a plurality of said oxidizing agent administration events, comprises intravaginal administration of the oxidizing agent contained in a said essential metal administration unit of the kit of any one of paragraphs 44-77.

142. The method of any one of paragraphs 131-141, wherein at least one said oxidizing agent administration event, and preferably each of a plurality of said oxidizing agent administration events, comprises administration of the oxidizing agent contained in a said oxidizing agent administration unit of the kit of any one of paragraphs 66-68.

143. The method of any one of paragraphs 131-142, wherein the oxidizing agent is hydrogen peroxide, and optionally hydrogen peroxide in the form of hydrogen peroxide-urea (urea peroxide).

### Treatment Regimens—Acidification Agent

144. The method of any one of paragraphs 86-143, wherein the treatment regimen comprises at least one acidification agent administration event, and preferably a plurality of acidification agent administration events, each said acidification agent administration event comprising intravaginal administration of acidification agent, and optionally at least one or each of a plurality of said acidification agent administration events is contemporaneous with an administration event selected from the group consisting of a said probiotic administration event of any one of paragraphs 94-105, a said lactoferrin administration event of any one of paragraphs 106-119, a said lipocalin-2 administration event of any one of paragraphs 120-130, a said oxidizing agent administration event of any one of paragraphs 131-143 and combinations thereof.

145. The method of paragraph 144, wherein at least one said acidification agent administration event, and optionally each of a plurality of said acidification agent administration events, is contemporaneous with a said metal administration event.

146. The method of either one of paragraph 144 or paragraph 145, wherein at least one said acidification agent administration event, and optionally each of a plurality of said acidification agent administration events, occurs after a said metal administration event, and further optionally on a different day than occurrence of a said metal administration event.

147. The method of any one of paragraphs 144-146, wherein each said acidification agent administration event comprises intravaginally administering a dose of acidification agent in an amount of at least 100 milligrams, and optionally at least 200 milligrams.

148. The method of any one of paragraphs 144-147, wherein each said acidification agent administration event comprises intravaginally administering a dose of acidification agent in amount of up to 600 milligrams.

149. The method of any one of paragraphs 144-148, wherein the treatment regimen comprises at least a minimum number of the acidification agent administration events selected from the group consisting of 2, 3, 4, 5, 7, 10 and 14, and optionally comprising a maximum number of the acidification agent administration events selected from the group consisting of 100, 50, 30 and 15; and with one preferred range for the number of the acidification agent administration events being in a range of from 7 to 30.

150. The method of paragraph 149, comprising a plurality of the acidification agent administration events and wherein the acidification agent administration events are at a frequency of not more than twice per day, preferably not more than once per day;
optionally, the acidification agent administration events are at a frequency of not less than once per week and preferably not more than once every 3 days; and
one preferred frequency for the metal administration events is once per day.

151. The method of either one of paragraph 149 or paragraph 150, wherein at least one said acidification agent administration event, and preferably at least each of the minimum number of the acidification agent administration events, occurs during a time commencing no earlier than one day prior to a first said metal administration event of the treatment regimen and ending not more than one day after a last said metal administration event of the treatment regimen.

152. The method of any one of paragraphs 144-151, wherein at least one said acidification agent administration event, and preferably each of a plurality of said acidification agent administration events, comprises intravaginal administration of the acidification agent contained in a composition according to any one of paragraphs 1-43.3.

153. The method of any one of paragraphs 144-152, wherein at least one said acidification agent administration event, and preferably each of a plurality of said acidification agent administration events, comprises intravaginal administration of the acidification agent contained in a said essential metal administration unit of the kit of any one of paragraphs 44-77.

154. The method of any one of paragraphs 144-153, wherein at least one said acidification agent administration event, and preferably each of a plurality of said acidification agent administration events, comprises administration of the acidification agent contained in a said acidification agent administration unit of the kit of any one of paragraphs 71-73.

154.1 The method of any one of paragraphs 144-154, wherein a said acidification administration event, or a series of sequential acidification administration events, reduces pH of the vaginal environment to pH 4.5 or lower, and preferably not below pH 3.0.

155. The method of any one of paragraphs 144-154.1, wherein the acidification agent is selected from the group consisting of boric acid, lactic acid and a lactate (e.g., calcium lactate or magnesium lactate), preferably selected from the group consisting of lactic acid and a lactate, and more preferably the acidification agent is a lactate.

### Treatment Regimens—Carbohydrate Source

156. The method of any one of paragraphs 86-155, wherein the treatment regimen comprises at least one carbohydrate source administration event, and preferably a plurality of carbohydrate source administration events, each said carbohydrate source administration event comprising intravaginal administration of a carbohydrate source to support growth of D-lactic acid-producing Lactobacillus, and optionally at least one or each of a plurality of said carbohydrate source administration events is contemporaneous with an administration event selected from the group consisting of a said probiotic administration event of any one of paragraphs 94-105, a said lactoferrin administration event of any one of paragraphs 106-119, a said lipocalin-2 administration event of any one of paragraphs 120-130, a said oxidizing agent administration event of any one of paragraphs 131-143, a said acidification administration event of any one of paragraphs 144-155, and combinations thereof.

157. The method of paragraph 156, wherein at least one said carbohydrate source administration event, and optionally each of a plurality of said carbohydrate source administration events, is contemporaneous with a said metal administration event.

158. The method of either one of paragraph 156 or paragraph 157, wherein at least one said carbohydrate source administration event, and optionally each of a plurality of said carbohydrate source administration events, occurs after a said metal administration event, and further optionally on a different day than occurrence of a said metal administration event.

159. The method of any one of paragraphs 156-158, wherein each said carbohydrate source administration event comprises intravaginally administering a dose of the carbohydrate source and as a consequence increasing a concentration of the carbohydrate source in vaginal fluid by at least 0.25% wt/vol.

160. The method of any one of paragraphs 156-159, wherein each said carbohydrate source administration event comprises intravaginally administering a dose of the carbohydrate source and as a consequence increasing a concentration of the carbohydrate source in vaginal fluid by not more than 3% wt/vol.

161. The method of any one of paragraphs 156-160, wherein the treatment regimen comprises at least a minimum number of the carbohydrate source administration events selected from the group consisting of 2, 3, 4, 5, 7, 10 and 14, and optionally comprising a maximum number of the carbohydrate source administration events selected from the group consisting of 100, 50, 30 and 15; and with one preferred range for the number of the carbohydrate source administration events being in a range of from 7 to 30.

162. The method of paragraph 161, comprising a plurality of the carbohydrate source administration events and wherein the carbohydrate source administration events are at a frequency of not more than twice per day, preferably not more than once per day;
optionally, the carbohydrate source administration events are at a frequency of not less than once per week and preferably not more than once every 3 days.

163. The method of either one of paragraph 161 or paragraph 162, wherein at least one said carbohydrate source administration event, and preferably at least each of the minimum number of the carbohydrate source administration events, occurs during a time commencing no earlier than one day prior to a first said metal administration event of the treatment regimen and ending not more than one day after a last said metal administration event of the treatment regimen.

164. The method of any one of paragraphs 156-163, wherein at least one said carbohydrate source administration event, and preferably each of a plurality of said carbohydrate source administration events, comprises intravaginal administration of the carbohydrate source contained in a composition according to any one of paragraphs 1-43.3.

165. The method of any one of paragraphs 156-164, wherein at least one said carbohydrate source administration event, and preferably each of a plurality of said carbohydrate source administration events, comprises intravaginal administration of the carbohydrate source contained in a said essential metal administration unit of the kit of any one of paragraphs 44-77.

166. The method of any one of paragraphs 156-165, wherein at least one said carbohydrate source administration event, and preferably each of a plurality of said carbohydrate source administration events, comprises administration of the carbohydrate source contained in a said carbohydrate source administration unit of the kit of any one of paragraphs 73.3-73.5.

167. The method of any one of paragraphs 156-166, wherein the carbohydrate source is a sugar with the sugar optionally comprising a member selected from the group consisting of sucrose and lactose, and preferably the carbohydrate source comprises lactose.

### Treatment Regimens—Miscellaneous

168. The method of any one of paragraphs 86-167, comprising a plurality of said metal administration events

169. The method of paragraph 168, wherein a said dose of the metal is the same for two or more of the metal administration events.

170. The method of paragraph 169, wherein a said dose of metal is different for two or more of the metal administration events.

171. The method of any one of paragraphs 86-170, wherein at least one said metal administration event comprises intravaginal administration of both manganese and zinc, optionally with the manganese and zinc administered in a same delivery composition and alternatively optionally with the manganese and the zinc in contemporaneously delivered different delivery compositions, and in either case each said delivery composition can optionally be in the form of the composition of any one of paragraphs 1-43.3 and/or each said delivery composition can be in a metal administration unit of the kit of any one of paragraphs 44-77.

172. The method of any one of paragraphs 86-171, wherein at least one said dose of essential metal, preferably each of plurality doses of essential metal and optionally each and every said dose of essential metal, comprises manganese in an amount selected from the group consisting of at least 2 micrograms, at least 5 micrograms, at least 10 micrograms, at least 20 micrograms, at least 30 micrograms and at least 50 micrograms.

173. The method of any one of paragraphs 86-172, wherein at least one said dose of essential metal, preferably each of plurality doses of essential metal and optionally each and every said dose of essential metal, comprises manganese in up to an amount selected from the group consisting of 2000 micrograms and 1000 micrograms.

174. The method of any one of paragraphs 86-173, wherein at least one said dose of essential metal, preferably each of plurality doses of essential metal and optionally each and every said dose of essential metal, comprises zinc in an amount of at least 4 micrograms, at least 10 micrograms, at least 25 micrograms, at least 50 micrograms of zinc or at least 100 micrograms.

175. The method of any one of paragraphs 86-174, wherein at least one said dose of essential metal, preferably each of plurality doses of essential metal and optionally each and every said dose of essential metal, comprises zinc in an amount up to 5000 micrograms.

175.1 The composition, kit or method of any one of paragraphs 1-175, wherein the vaginal dysbiotic condition is bacterial vaginosis.

175.2 The composition, kit or method of any one of paragraphs 1-175, wherein the vaginal dysbiotic condition is pre-bacterial vaginosis.

### Methods of Analysis

176. A method of analyzing vaginal microbiota condition, the method comprising assaying vaginal fluid of a person to determine a level of calprotectin in the vaginal fluid, and optionally the assaying is according to the method of any one of paragraphs 184.1-184.19.3.

177. A method of paragraph 176, wherein the assaying comprises subjecting to analysis a neat vaginal fluid sample, optionally of known quantity, preferably a known liquid volume, as received for the assaying.

178. The method of paragraphs 177, wherein a volume of the vaginal fluid sample as received for the assaying is not larger than 100 microliters, preferably not larger than 50 microliters and more preferably not larger than 30 microliters, and even more preferably not larger than 20 microliters;
preferably, the volume of the vaginal fluid sample as received for the assaying comprises at least 3 microliters, preferably at least 5 microliters and more preferably at least 7 microliters; and
one preferred size for the volume of the vaginal fluid sample as received for the assaying is about 10 microliters.

179. The method of either one of paragraph 177 or paragraph 178, wherein the assaying comprises;
receiving the vaginal fluid sample of known quantity for the assaying contained in a hermetically sealed container;
unsealing the hermetically sealed container and accessing the vaginal fluid sample to perform the analysis.

180. The method of paragraph 179, wherein the hermetically sealed container comprises an internal fluid containment volume of no larger than 100 microliters.

181. The method of either one of paragraph 179 or paragraph 180, wherein the hermetically sealed container comprises an internal fluid containment volume of no larger than 3 times, preferably no larger than 2 times and more preferably no larger than 1.5 times a volume of the vaginal fluid sample contained in the hermetically sealed container.

182. The method of any one of paragraphs 176-181, comprising receiving in frozen form the vaginal fluid sample for the assaying.

183. The method of any one of paragraphs 176-182, wherein the assaying comprises an analytical technique selected from the group consisting of ELISA or other immunoassay methods, high performance liquid chromatography (HPLC)-based methods and combinations thereof.

184. The method of any one of paragraphs 176-183, wherein the assaying comprises determining a concentration of calprotectin in the vaginal fluid, and preferably in a said vaginal fluid sample as received for the assaying.

184.1 A method for assaying a vaginal fluid sample for calprotectin, preferably a neat vaginal fluid sample, the method comprising analyzing the vaginal fluid sample for total extracellular calprotectin in the vaginal fluid sample. By total extracellular calprotectin it is meant extracellular calprotectin in the vaginal fluid sample whether determined as a single amount or as multiple partial amounts associated with different parts of the vaginal fluid sample that if added together represent a total amount of extracellular calprotectin, and whether determined as an absolute total quantity or multiple absolute partial quantities or as a total concentration or multiple partial concentrations or otherwise.

184.2 The method of paragraph 184.1, comprising dispersing, and preferably dissolving, at least a portion of the extracellular calprotectin in an aqueous liquid composition, and wherein the aqueous liquid composition is at a pH that is higher than a pH of the vaginal fluid sample.

184.3 The method of either one of paragraph 184.1 or paragraph 184.2, comprising dispersing, and preferably dissolving, at least a portion of the extracellular calprotectin in an aqueous liquid composition, and wherein the aqueous liquid composition is at a pH of at least pH 6, preferably at least pH 6.5 more preferably at least pH 7.0 and even more preferably at least pH 7.2.

184.4 The method of any one of paragraphs 184.1-184.3, comprising dispersing, and preferably dissolving, at least a portion of the extracellular calprotectin in an aqueous liquid composition, and wherein the aqueous liquid composition is at a pH of not larger than pH 8.5, preferably not larger than pH 8.0 more preferably not large than pH 7.8 and even more preferably not larger than 7.6, and with one preferred range being from pH 6.5 to pH 8.0, another preferred range being from pH 7.0 to pH 8.0, another preferred range being from pH 7.2 to pH 7.8 and yet another preferred range being from pH 7.2 to pH 7.6.

184.5 The method of any one of paragraphs 184.2-184.4, comprising, after the dispersing, analyzing the aqueous liquid composition to determine a level of the extracellular calprotectin in the aqueous liquid composition, and optionally wherein the analyzing the aqueous liquid composition comprises use of at least one analytical technique selected from the group consisting of ELISA or other immunoassay methods, high performance liquid chromatography (HPLC)-based methods and combinations thereof.

184.6 The method of any one of paragraphs 184.1-184.5, comprising centrifuging the vaginal fluid sample to prepare at least a separated solids fraction and a separated liquid fraction.

184.7 The method of paragraph 184.6, wherein the centrifuging comprises centrifuging a vaginal sample collection device to remove the vaginal fluid sample from the sample collection device to prepare the separated solids fraction and separated liquid fraction, and optionally the sample collection device comprises a swab tip.

184.8 The method of paragraphs 184.7, comprising after the centrifuging, washing the solids fraction with an aqueous wash liquid to disperse in, and preferably to dissolve in, the aqueous wash liquid least a portion, preferably a majority and more preferably substantially all, of the extracellular calprotectin of the fluid sample associated with the solids fraction after the centrifuging, and to prepare a loaded aqueous wash liquid comprising dispersed, and preferably dissolved, extracellular calprotectin removed from the solids fraction, and optionally the aqueous wash liquid and/or the loaded aqueous wash liquid is the aqueous liquid composition, or has a pH of the aqueous liquid composition, of any one of paragraphs 184.2-184.5.

184.9 The method of any one of paragraphs 184.1-184.8, comprising determining a concentration value for total assayed extracellular calprotectin in the vaginal fluid sample.

184.9.1 The method of any one of paragraphs 184.1-184.9, comprising determining a concentration value for assayed extracellular calprotectin from an identified portion of the vaginal fluid sample, optionally a separated solids portion and/or a separated liquid portion.

184.9.2 The method of any one of paragraphs 184.1-184.9.1, comprising measuring a quantity of a separated solids fraction of the vaginal fluid sample and using the measured quantity of the separated solids fraction in a determination of a concentration value for assayed extracellular calprotectin in all or an identified portion or portions of the vaginal fluid sample.

184.9.3 The method of any one of paragraphs 184.1-184.9.2, comprising measuring a quantity of a separated liquid fraction of the vaginal fluid sample and using the measured quantity of the separated liquid fraction in a determination of a concentration value for assayed extracellular calprotectin in all or an identified portion or portions of the vaginal fluid sample.

184.10 A method for assaying a vaginal fluid sample for calprotectin, preferably a neat vaginal fluid sample, the method comprising analyzing the vaginal fluid sample for intracellular calprotectin, and optionally for total intracellular calprotectin in the vaginal fluid sample;
and, optionally, the vaginal fluid sample is the vaginal fluid sample of any one of paragraphs 18.1-184.9.3 and the method also comprises performing the method of any one of paragraphs 184.1-184.9.3

184.11 The method of paragraph 184.10, comprising lysing at least a portion of cells, and preferably substantially all of the cells, of the vaginal fluid sample to release intracellular calprotectin for recovery.

184.12 The method of either one of paragraph 184.10 or paragraph 184.11, wherein the at least a portion of the cells, and optionally substantially all of the cells, subjected to the lysing are in a separated solids fraction from centrifugation of the vaginal fluid sample.

184.13 The method of paragraph 184.12, comprising centrifuging the vaginal fluid sample to prepare at least the separated solids fraction and a separated liquid fraction, and optionally the centrifuging is according to the method of paragraph 184.6 or 184.7.

184.14 The method of either one of paragraphs 184.12 or paragraph 184.13, wherein the lysing comprises treating the solids fraction with a lysis solution, optionally an aqueous lysis solution, to lyse the at least a portion, and preferably substantially all, of the cells in the solids fraction.

184.15 The method of either one of paragraph 184.13 or 184.14, wherein the centrifuging comprises centrifuging a vaginal sample collection device to remove the vaginal fluid sample from the sample collection device to prepare the separated solids fraction and separated liquid fraction, and optionally the sample collection device comprises a swab tip.

184.16 The method of any one of paragraphs 184.10-184.15, comprising dispersing, and preferably dissolving, at least a portion of the released intracellular calprotectin, and preferably substantially all of the released intracellular calprotectin, in an aqueous extraction liquid, and optionally the aqueous extraction liquid comprises aqueous lysis solution contacted with at least a portion of the vaginal fluid sample to lyse the at least a portion of the cells, and optionally the lysis solution according to paragraph 184.14.

184.17 The method of paragraph 184.16, comprising after the dispersing at least a portion of the released intracellular calprotectin in an aqueous extraction liquid, analyzing the aqueous extraction liquid to determine a level of the extracellular calprotectin in the aqueous extraction liquid, and optionally wherein the analyzing the aqueous extraction liquid comprises use of at least one analytical technique selected from the group consisting of ELISA or other immunoassay methods, high performance liquid chromatography (HPLC)-based methods and combinations thereof.

184.18 The method of any one of paragraphs 184.10-184.17, comprising determining a concentration value for total assayed intracellular calprotectin in the vaginal fluid sample.

184.18.1 The method of any one of paragraphs 184.10-184.18, comprising determining a concentration value for assayed intracellular calprotectin from an identified portion of the vaginal fluid sample, optionally a separated solids portion.

184.18.2 The method of any one of paragraphs 184.10-184.18.1, comprising measuring a quantity of a separated solids fraction of the vaginal fluid sample and using the measured quantity of the separated solids fraction in a determination of a concentration value for assayed intracellular calprotectin in all or an identified portion or portions of the vaginal fluid sample.

184.18.3 The method of any one of paragraphs 184.10-184.18.2, comprising measuring a quantity of a separated liquid fraction of the vaginal fluid sample and using the measured quantity of the separated liquid fraction in a determination of a concentration value for assayed intracellular calprotectin in all or an identified portion or portions of the vaginal fluid sample.

184.19, The method of any one of paragraphs 184.1-184.18.3, comprising determining a concentration value for total assayed calprotectin in the vaginal fluid sample.

184.19.1 The method of any one of paragraphs 184.1-184.19, comprising determining a concentration value for total assayed calprotectin from an identified portion of the vaginal fluid sample, optionally a separated solids portion and/or a separated liquid portion.

184.19.2 The method of any one of paragraphs 184.1-184.19.1, comprising measuring a quantity of a separated solids fraction of the vaginal fluid sample and using the measured quantity of the separated solids fraction in a determination of a concentration value for total assayed calprotectin in all or an identified portion or portions of the vaginal fluid sample.

184.19.3 The method of any one of paragraphs 184.1-184.19.2, comprising measuring a quantity of a separated liquid fraction of the vaginal fluid sample and using the measured quantity of the separated liquid fraction in a determination of a concentration value for total assayed calprotectin in all or an identified portion or portions of the vaginal fluid sample.

185. The method of any one of paragraphs 176-184.19.3, wherein the assaying further comprises determining a level, and preferably a concentration, of at least one other analyte in the vaginal fluid, wherein the one other analyte is indicative of vaginal microbiota health.

186. The method of paragraphs 185, wherein the assay comprises determining both the calprotectin level and the level of the at least one other analyte in the vaginal fluid from analysis of a single said vaginal fluid sample received for the assaying or from analysis of multiple contemporaneously obtained said vaginal fluid samples received for the assaying, and preferably from analysis of a single said vaginal fluid sample.

187. The method of either one of paragraph 185 or paragraph 186, wherein the at least one other analyte comprises a member selected from the group consisting of D-lactate; L-Lactate; acetate and combinations thereof.

188. The method paragraph 187, comprising determining a concentration ratio between D-lactate level and L-lactate level.

189. The method of any one of paragraphs 176-188, comprising correlating the level of calprotectin, and optionally also of one or more other analytes, to vaginal microbiota assay information of target bacteria corresponding to the level of calprotectin, wherein the vaginal microbiota assay information comprising whether or not each said target bacteria is detected and quantification indications for detected target bacteria of at least relative abundances of the detected target bacteria, wherein the target bacteria comprise:
(a) at least one first target bacteria comprising one or more D-lactic acid-producing Lactobacillus species; and
(b) at least one second target bacteria selected from the group consisting of bacteria associated with bacterial vaginosis, Lactobacillus iners and combinations thereof, and optionally the bacteria associated with bacterial vaginosis comprises at least one member selected from the group consisting of Gardnerella vaginalis, Atopobium vaginae, one or more Prevotella species, one or more Mobiluncus species and combinations thereof, and preferably the bacteria indicative of bacterial vaginosis comprises at least Gardnerella vaginalis.

190. The method of paragraph 189, wherein the second target bacteria comprise both Gardnerella vaginalis and Lactobacillus iners.

191. The method of either one of paragraph 189 or paragraph 190, wherein the first target bacteria comprise at least one member selected from the group consisting of the group consisting of Lactobacillus crispatus, Lactobacillus jensenii, Lactobacillus johnsonii, Lactobacillus gasseri, Lactobacillus helveticus, Lactobacillus rhamnosus, Lactobacillus reuteri, Lactobacillus casei, Lactobacillus plantarum and combinations thereof; preferably the first target bacteria comprise at least one member selected from the group consisting of Lactobacillus crispatus, Lactobacillus jensenii, Lactobacillus gasseri and combinations thereof; and more preferably the first target bacteria comprise Lactobacillus crispatus.

192. The method of any one of paragraphs 189-191, wherein the first target bacteria comprise Lactobacillus crispatus.

193. The method any one of paragraphs 189-192, wherein the first target bacteria comprise Lactobacillus gasseri.

194. The method of any one of paragraphs 189-193, wherein the first target bacteria comprise Lactobacillus jensenii.

195. The method of any one of paragraphs 189-194, wherein the quantification indications of the second assay results include an absolute quantification indication for each said detected target bacteria.

196. The method of any one of paragraphs 189-1954, comprising performing a microbiota assay on a vaginal fluid sample to generate the vaginal microbiota assay information, wherein the vaginal fluid sample is a same vaginal fluid sample or a contemporaneous vaginal fluid sample used for the assaying to determine a level of calprotectin.

### Methods of Diagnosis

197. A method of diagnosing a vaginal dysbiotic condition in a person, the method comprising:
evaluating an assayed vaginal calprotectin level in vaginal fluid of the person, and optionally the assayed vaginal calprotectin level is from a method according to any one of paragraphs 176-196; and
diagnosing the vaginal dysbiotic condition based at least in part on the evaluating determining that there is an elevated level of vaginal calprotectin, and optionally with the elevated level of calprotectin corresponding with at least one other positive indication of vaginal bacterial dysbiosis.

198. The method of paragraph 197, comprising diagnosing the vaginal dysbiotic condition when the assayed calprotectin level is at a concentration of calprotectin in vaginal fluid at or above a concentration threshold for calprotectin, preferably the threshold is at least 3 micromoles per liter and even more preferably the threshold is at least 5 micromoles per liter.

199. The method of either one of paragraph 197 or paragraph 198, wherein the evaluating comprises correlating the assayed vaginal calprotectin level with at least one other indication of possible vaginal bacterial dysbiosis.

200. The method of paragraph 199, wherein the at least one other indication comprises vaginal microbiota assay information, optionally the vaginal microbiota assay information of any one of paragraphs 189-196.

201. The method of paragraph 200, comprising diagnosing the vaginal dysbiotic condition based at least in part on the presence of a deficiency of at least one D-Lactic acid-producing Lactobacillus species in the vaginal microbiota; and
optionally the at least one D-lactic acid-producing Lactobacillus comprises a member selected from the group consisting of Lactobacillus crispatus, Lactobacillus jensenii, Lactobacillus johnsoni, Lactobacillus gasseri, Lactobacillus helveticus, Lactobacillus rhamnosus, Lactobacillus reuteri, Lactobacillus casei, Lactobacillus plantarum and combinations thereof; preferably the at least one D-lactic acid-producing Lactobacillus comprises a member selected from the group consisting of Lactobacillus crispatus, Lactobacillus jensenii, Lactobacillus gasseri and combinations thereof; and more preferably the at least one D-lactic acid-producing Lactobacillus comprises Lactobacillus crispatus.

202. The method of either one or paragraph 200 or paragraph 201, comprising diagnosing the vaginal dysbiotic condition based at least in part on the presence of a relative over-abundance of bacteria associated with bacterial vaginosis, optionally including a member selected from the group consisting of Lactobacillus iners, Gardnerella vaginalis, Atopobium vaginae, one or more Prevotella species, one or more Mobiluncus species and combinations thereof, and preferably the bacteria indicative of bacterial vaginosis comprises at least Gardnerella vaginalis.

203. The method of any one of paragraphs 200-202, comprising diagnosing the vaginal dysbiotic condition based at least in part on a deficiency of first bacteria comprising at least one D-lactic acid-producing Lactobacillus relative to second bacteria comprising at least one bacteria associated with bacterial vaginosis;
optionally the at least one D-lactic acid-producing Lactobacillus comprises a member selected from the group consisting of Lactobacillus crispatus, Lactobacillus jensenii, Lactobacillus johnsoni, Lactobacillus gasseri, Lactobacillus helveticus, Lactobacillus rhamnosus, Lactobacillus reuteri, Lactobacillus casei, Lactobacillus plantarum and combinations thereof; preferably the at least one D-lactic acid-producing Lactobacillus comprises a member selected from the group consisting of Lactobacillus crispatus, Lactobacillus jensenii, Lactobacillus gasseri and combinations thereof; and more preferably the at least one D-lactic acid-producing Lactobacillus comprises Lactobacillus crispatus;
and optionally the member selected from the group consisting of Lactobacillus iners, Gardnerella vaginalis, Atopobium vaginae, one or more Prevotella species, one or more Mobiluncus species and combinations thereof, and preferably the bacteria indicative of bacterial vaginosis comprises at least Gardnerella vaginalis.

204. The method of any one of paragraphs 199-203, wherein the at least one other indication comprises a member selected from the group consisting of;
relative presence or absence of D-lactate in vaginal fluid;
relative presence or absence of L-lactate in vaginal fluid, and optionally the relative abundance of L-lactate to D-lactate;
relative presence or absence of acetate in vaginal fluid; and
combinations thereof.

205. The method of any one of paragraphs 199-204, wherein the at least one other indication comprises at least one member selected from the group consisting of:
a patient history of recurring bacterial vaginosis;
abnormal vaginal discharge;
a positive whiff test for malodorous vaginal fluid;
a vaginal itching;
a burning during urination;
a milky, homogenous vaginal discharge;
a vaginal pH above pH 4.5; and
combinations thereof.

206. The method of any one of paragraphs 197-205, wherein the vaginal dysbiotic condition is bacterial vaginosis.

207. The method of any one of paragraphs 197-205, wherein the vaginal dysbiotic condition is pre-bacterial vaginosis.

208. The method of any one of paragraphs 197-207, wherein the diagnosing comprises diagnosing a relative severity of the vaginal dysbiotic condition based at least in part on the magnitude of elevation of the vaginal calprotectin, and optionally also based at least in part on one more or more of the at least one other indication of paragraphs 199-205; wherein different levels of diagnosed severity are indicative of different therapeutic treatment protocols to treat the vaginal dysbiotic condition.

### Methods of Discontinuing Treatment

209. A method for discontinuing therapeutic treatment of a vaginal dysbiotic condition, wherein the therapeutic treatment comprises a treatment regimen with a plurality of temporally spaced administration events each including administration, preferably by intravaginal administration, of a dose of at least one therapeutic agent to treat the vaginal dysbiotic condition, the method comprising:
monitoring a level of calprotectin in vaginal fluid; and
discontinuing the therapeutic treatment after the level of calprotectin in the vaginal fluid has dropped to below a threshold level.

210. The method of paragraph 209, comprising discontinuing the therapeutic treatment after an assay of vaginal fluid for vaginal microbiota indicates the vaginal microbiota is dominated by D-lactic acid producing Lactobacillus.

211. The method of either one of paragraph 209 or paragraph 210, comprising discontinuing the therapeutic treatment after an assay of vaginal fluid for D-lactate, and optionally also for L-lactate, indicates a level of D-lactate in vaginal fluid, and optionally relative to L-lactate in vaginal fluid, indicative of a vaginal microbiota dominated by D-lactic acid-producing Lactobacillus.

212. The method of any one of paragraphs 209-211, comprising discontinuing the therapeutic treatment after an assay of vaginal fluid for acetate in vaginal fluid indicates an absence or low level of acetate indicative of a vaginal microbiota not being dominated by bacteria associated with bacterial vaginosis (e.g., Gardnerella vaginalis, Atopobium vaginae, Prevotella species, Mobiluncus species).

213. The method of any one of paragraphs 209-212, wherein the therapeutic treatment is according to the method of any one of paragraphs 78-196.

The foregoing description of the present invention and various aspects thereof has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

The description of a feature or features in a particular combination do not exclude the inclusion of an additional feature or features in a variation of the particular combination. Processing steps and sequencing are for illustration only, and such illustrations do not exclude inclusion of other steps or other sequencing of steps to an extent not necessarily incompatible. Additional steps may be included between any illustrated processing steps or before or after any illustrated processing step to an extent not necessarily incompatible.

The terms “comprising”, “containing”, “including” and “having”, and grammatical variations of those terms, are intended to be inclusive and nonlimiting in that the use of such terms indicates the presence of a stated condition or feature, but not to the exclusion of the presence also of any other condition or feature. The use of the terms “comprising”, “containing”, “including” and “having”, and grammatical variations of those terms in referring to the presence of one or more components, subcomponents or materials, also include and is intended to disclose the more specific embodiments in which the term “comprising”, “containing”, “including” or “having” (or the variation of such term) as the case may be, is replaced by any of the narrower terms “consisting essentially of” or “consisting of” or “consisting of only” (or any appropriate grammatical variation of such narrower terms). For example, a statement that something “comprises” a stated element or elements is also intended to include and disclose the more specific narrower embodiments of the thing “consisting essentially of” the stated element or elements, and the thing “consisting of” the stated element or elements. Examples of various features have been provided for purposes of illustration, and the terms “example”, “for example” and the like indicate illustrative examples that are not limiting and are not to be construed or interpreted as limiting a feature or features to any particular example. The term “at least” followed by a number (e.g., “at least one”) means that number or more than that number. The term at “at least a portion” means all or a portion that is less than all. The term “at least a part” means all or a part that is less than all.

What is claimed is:

1. A composition for use in the treatment of a vaginal dysbiotic condition with an elevated level of calprotectin in vaginal fluid, the composition being formulated for intravaginal administration and the composition comprising:
a dose of essential metal for growth of vaginal flora Lactobacillus comprising at least one D-lactic acid-producing Lactobacillus, the essential metal being in a pharmaceutically acceptable form for intravaginal administration and the essential metal comprising manganese, zinc or a combination of manganese and zinc; and
wherein the dose of essential metal is in an amount to satisfy the following condition:
$$( ( 0.5 \times {Q}_{Zn} ) + {Q}_{Mn} ) \geq ( 0.001 \times K \times {C}_{ECp} )$$
wherein
Q_{Zn} is zero or a positive number equal to an amount of zinc in micromoles;
Q_{Mn} is zero or a positive number equal to an amount of manganese in micromoles;
C_{ECp} is a molar concentration of elevated calprotectin in vaginal fluid, in micromoles per liter;
K is a positive number of 1 or greater; and
at least one of Q_{Zn} and Q_{Mn} is a positive number.

2. The composition of claim 1, wherein C_{ECp} is at least 3.

3. The composition of either one of claim 1 or claim 2, wherein the dose of essential metal is in an amount to satisfy the following condition:
$$( ( 0.5 \times {Q}_{Zn} ) + {Q}_{Mn} ) \geq ( 0.1 \times K )$$
wherein
Q_{Zn} is the amount of zinc in micromoles;
Q_{Mn} is the amount of manganese in micromoles; and
K is a positive number of 1 or greater.

4. The composition of any one of claims 1-3, wherein K is 3.

5. The composition of any one of claims 1-4, comprising manganese in an amount of at least 2 micrograms.

6. The composition of any one of claims 1-5, comprising manganese in an amount of at least 50 micrograms.

7. The composition of any one of claims 1-6, comprising manganese up to an amount of 2000 micrograms.

8. The composition of any one of claims 1-7, wherein the composition comprises zinc and the composition is in the absence of manganese.

9. The composition of any one of claims 1-8, comprising zinc in an amount of at least 4 micrograms.

10. The composition of any one of claims 1-9, comprising zinc in an amount of at least 100 micrograms.

11. The composition of any one of claims 1-10, comprising zinc in an amount up to 5000 micrograms.

12. The composition of any one of claims 1-7, wherein the composition comprises manganese and is in the absence of zinc.

13. The composition of any one of claims 1-12, comprising a probiotic, and wherein the probiotic comprises at least one D-lactic acid-producing Lactobacillus species.

14. The composition of any one of claims 1-13, comprising lactoferrin;
wherein the lactoferrin is iron-depleted lactoferrin comprising a level of saturation with bound iron, in increasing order of preference, of not larger than 15 percent, not larger than 10 percent, not larger than 5 percent, not larger than 4 percent, not larger than 3 percent, not larger than 2 percent and most preferably not larger than 1 percent.

15. The composition of any one of claims 1-14, comprising lipocalin-2 in an amount of from 0.3 milligrams to 30 milligrams.

16. The composition of any one of claims 1-15, comprising an oxidizing agent in an amount formulated in the composition with a release profile to provide an increase in oxidation-reduction potential (ORP), relative to a standard hydrogen electrode, of vaginal fluid of at least 50 mV.

17. The composition of any one of claims 1-16, comprising an acidification agent to help lower pH of the vaginal environment, wherein the acidification agent is in an amount of at least 100 milligrams and up to 600 milligrams.

18. The composition of any one of claims 1-17, comprising a carbohydrate source for the D-lactic acid producing Lactobacillus, wherein the carbohydrate source is in an amount formulated in the composition with a release profile to provide a concentration of the carbohydrate source in vaginal fluid in a range of from 0.25% wt/vol to 3% wt/vol.

19. The composition of any one of claims 1-18, comprising a plurality of said doses of essential metal, and wherein the composition is divisible into a plurality of separate aliquots each comprising a said dose of essential metal.

20. The composition of any one of claims 1-19, wherein the composition is formulated to release the essential metal of the dose after intravaginal administration for therapeutic effect over a period of time of at least 12 hours.

21. A kit for use in the treatment of a vaginal dysbiotic condition with an elevated level of vaginal calprotectin, the kit comprising;
a plurality of administration units, the plurality of administration units comprising a plurality of essential metal administration units formulated for intravaginal administration, each said essential metal administration unit comprising a dose of essential metal for growth of at least one vaginal flora Lactobacillus comprising at least one D-lactic acid-producing Lactobacillus;
the essential metal being in a pharmaceutically acceptable form; and
the essential metal comprising manganese, zinc or a combination of manganese and zinc; and
the dose of essential metal is in an amount to satisfy the following condition:
$$( ( 0.5 \times {Q}_{Zn} ) + {Q}_{Mn} ) \geq ( 0.001 \times K \times {C}_{ECp} )$$
wherein
Q_{Zn} is zero or a positive number equal to an amount of zinc in micromoles;
Q_{Mn} is zero or a positive number equal to an amount of manganese in micromoles;
C_{ECp} is a molar concentration of elevated calprotectin in vaginal fluid, in micromoles per liter;
K is a positive number of 1 or greater; and
at least one of Q_{Zn} and Q_{Mn} is a positive number; and
wherein each said essential metal administration unit comprises a composition according to any one of claims 1-19.

22. A kit for use in the treatment of a vaginal dysbiotic condition with an elevated level of vaginal calprotectin, the kit comprising;
a plurality of administration units, the plurality of administration units comprising at least one essential metal administration unit, each said essential metal administration unit comprising a dose of essential metal for growth of at least one vaginal flora Lactobacillus comprising at least one D-lactic acid-producing Lactobacillus;
the essential metal being in a pharmaceutically acceptable form; and
the essential metal comprising manganese, zinc or a combination of manganese and zinc; and
the dose of essential metal is in an amount to satisfy the following condition:
$$( ( 0.5 \times {Q}_{Zn} ) + {Q}_{Mn} ) \geq ( 0.001 \times K \times {C}_{ECp} )$$
wherein
Q_{Zn} is zero or a positive number equal to an amount of zinc in micromoles;
Q_{Mn} is zero or a positive number equal to an amount of manganese in micromoles;
C_{ECp} is a molar concentration of elevated calprotectin in vaginal fluid, in micromoles per liter;
K is a positive number of 1 or greater; and
at least one of Q_{Zn} and Q_{Mn} is a positive number.

23. The kit of either one of claim 21 or claim 22, comprising a number of said essential metal administration units in a range of from 2 to 100.

24. The kit of any one of claims 21-23, comprising in at least one said administration unit a probiotic comprising at least one D-lactic acid-producing Lactobacillus species.

25. The kit of claim 24, wherein at least one of said essential metal administration units comprise the probiotic.

26. The kit of either one of claim 24 or claim 25, comprising at least one probiotic administration unit, being a said administration unit different than a said essential metal administration unit, and each said probiotic administration unit comprising the probiotic.

27. The kit of claim 26, wherein at least one said probiotic administration unit is formulated for oral administration.

28. The kit of either one of claim 26 or claim 27, wherein at least one said probiotic administration unit is formulated for intravaginal administration.

29. The kit of any one of claims 24-28, wherein each said administration unit comprising the probiotic comprises the probiotic in an amount of at least 10⁶ colony forming units (CFU).

30. The kit of any one of claims 21-29, comprising in at least one said administration unit an iron-depleted bovine lactoferrin, and wherein each said lactoferrin unit is formulated for intravaginal administration.

31. The kit of claim 30, comprising at least one lactoferrin administration unit, each said lactoferrin administration unit being a said administration unit different than a said essential metal administration unit, and each said lactoferrin administration unit comprising the iron-depleted bovine lactoferrin.

32. The kit of either one of claim 30 or claim 31, wherein each said administration unit comprising iron-depleted bovine lactoferrin comprises the iron-depleted bovine lactoferrin in an amount of from 10 milligrams to 500 milligrams; and
wherein the iron-depleted lactoferrin comprises a level of saturation with bound iron not larger than 5 percent.

33. The kit of any one of claims 21-32, comprising in at least one said administration unit lipocalin-2, and wherein each said administration unit comprising lipocalin-2 is formulated for intravaginal delivery.

34. The kit of claim 33, comprising at least one lipocalin-2 administration unit, each said lipocalin-2 administration unit being a said administration unit different than a said essential metal administration unit, and each said lipocalin-2 administration unit comprising lipocalin-2 in an amount of from 0.3 milligrams to 30 milligrams.

35. The kit of any one of claims 21-34, comprising in at least one said administration unit an oxidizing agent, and wherein each said administration unit comprising the oxidizing agent is formulated for intravaginal delivery.

36. The kit of claim 35, wherein the oxidizing agent comprises hydrogen peroxide in the form of hydrogen peroxide urea.

37. The kit of either one of claim 35 or claim 36, comprising at least one oxidizing agent administration unit, each said oxidizing administration unit being a said administration unit different than a said essential metal administration unit, and each said oxidizing agent administration unit comprising the oxidizing agent; and
wherein each said administration unit comprising the oxidizing agent comprises the oxidizing agent in an amount formulated in the administration unit with a release profile to provide an increase in oxidation-reduction potential (ORP), relative to a standard hydrogen electrode, of vaginal fluid of at least 50 mV.

38. The kit of any one of claims 21-37, comprising in at least one said administration unit an acidification agent to help lower pH of the vaginal environment, and wherein each said administration unit comprising the acidification agent is formulated for intravaginal delivery.

39. The kit of claim 38, comprising at least one acidification agent administration unit, each said acidification agent administration unit comprising the acidification agent, and wherein each said administration unit comprising the acidification agent comprises the acidification agent in an amount of from 100 milligrams to 500 milligrams.

40. The kit of either one of claim 38 or claim 39, wherein the acidification agent is selected from the group consisting of boric acid, lactic acid and a lactate.

41. The kit of any one claims 21-40, comprising in at least one said administration unit a carbohydrate source for the D-lactic acid producing Lactobacillus, and wherein each said administration unit comprising the carbohydrate source is formulated for intravaginal delivery.

42. The kit of claim 41, comprising at least one carbohydrate source administration unit, each said carbohydrate source administration unit comprising the carbohydrate source.

43. The kit of either one of claim 41 are claim 42, wherein each said administration unit comprising the carbohydrate source comprises carbohydrate source in an amount formulated in the composition with a release profile to provide a concentration of the carbohydrate source in vaginal fluid of from 0.25% wt/vol to 3% wt/vol.

44. The kit of any one of claims 21-43, comprising a common packaging enclosure containing the plurality of the administration units.

45. A method of treating a person for a vaginal dysbiotic condition with an analytically-determined, elevated calprotectin concentration (C_{ECp}) in vaginal fluid, the method comprising:
intravaginal administration to the person of essential metal comprising zinc, manganese or combinations of zinc and manganese sufficient to achieve an essential metal loading ratio in vaginal fluid of 1.2 or larger, wherein the essential metal loading ratio is calculated by the equation
$${R}_{EM} = ( ( 0.5 \times {C}_{Zn} ) + {C}_{Mn} ) / {C}_{ECp}$$
wherein,
R_{EM} is the essential metal loading ratio;
C_{Zn} is zero or a positive number equal to a concentration in micromoles per liter of zinc in the vaginal fluid; and
C_{Mn} is zero or a positive number equal to a concentration in micromoles per liter of manganese in the vaginal fluid;
C_{ECp} is the analytically-determined elevated calprotectin concentration in micromoles per liter; and
at least one of C_{Zn} and C_{Mn} is a positive number.

46. The composition, kit or method of any one of claims 1-45, wherein the vaginal dysbiotic condition is bacterial vaginosis.

47. The composition, kit or method of any one of claims 1-45, wherein the vaginal dysbiotic condition is pre-bacterial vaginosis.
